(19) United States
(12) Patent Application Publication
(10) Pub. No.: US 2015/0072968 A1
(43) Pub. Date: Mar. 12, 2015

(54) Treating Infections with Ceftolozane/Tazobactam in Subjects Having Impaired Renal Function

(71) Applicant: Calixa Therapeutics, Inc., Lexington, MA (US)

(72) Inventors: Gopal Krishna, Stow, MA (US); Gurudatt Chandorkar, Waltham, MA (US); Elham Hershberger, Lexington, MA (US); Benjamin Miller, Cambridge, MA (US); Alan Xiao, Lexington, MA (US)

(73) Assignee: Calixa Therapeutics, Inc., Lexington, MA (US)

(21) Appl. No.: 14/286,392

(22) Filed: May 23, 2014

Related U.S. Application Data
(60) Provisional application No. 61/875,358, filed on Sep. 9, 2013.
(60) Provisional application No. 61/883,579, filed on Sep. 27, 2013.
(60) Provisional application No. 61/978,625, filed on Apr. 11, 2014.
(60) Provisional application No. 61/984,299, filed on Apr. 25, 2014.
(60) Provisional application No. 61/988,085, filed on May 2, 2014.

(51) Int. Cl. A61K 31/546; A61K 31/4164; A61K 31/431

(52) U.S. Cl. CPC A61K 31/546; A61K 31/431; A61K 31/4164
USPC 514/192

(57) ABSTRACT

Disclosed is a method of administering cephalosporin/tazobactam to human patients with end stage renal disease undergoing hemodialysis and suffering from a complicated intra-abdominal infection or a complicated urinary tract infection.

### RELATED APPLICATIONS

This patent application claims the benefit of U.S. Provisional Patent Applications 61/875,358 (filed Sep. 9, 2013), 61/883,579 (filed Sep. 27, 2013), 61/978,625 (filed Apr. 11, 2014), 61/984,299 (filed Apr. 25, 2014), and 61/988,085 (filed May 2, 2014) each of which is incorporated herein by reference in its entirety.

### TECHNICAL FIELD

This disclosure relates to the administration of ceftolozane/tazobactam to human patients with impaired renal function.

### BACKGROUND

The cephalosporin (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate (also referred to as “ceftolozane”) is an antibacterial agent. The antibacterial activity of ceftolozane is believed to result from its interaction with penicillin binding proteins (PBPs) to inhibit the biosynthesis of the bacterial cell wall which acts to stop bacterial replication.

Ceftolozane can be combined (e.g., mixed) with a β-lactamase inhibitor (“BLI”), such as tazobactam. Tazobactam is a BLI against Class A and some Class C β-lactamases, with well-established in vitro and in vivo efficacy in combination with active β-lactam antibiotics. The combination of ceftolozane, or a pharmaceutically acceptable salt thereof and tazobactam or a pharmaceutically acceptable salt thereof in an amount providing a 2:1 weight ratio between the amount of ceftolozane active and tazobactam active is an antibiotic pharmaceutical composition (“CXA-201”) formulated for parenteral administration. CXA-201 displays potent antibacterial activity in vitro against common Gram-negative and selected Gram-positive organisms that cause complicated intra-abdominal infections or complicated urinary tract infections. Moreover, CXA-201 has demonstrated efficacy in clinical trials against these infections (See, e.g., Examples 4-5), and the recommended dosage for patients with normal kidney function [creatinine clearance (CrCL)>50 mL/min] is 1.5 grams of CXA-201 (1 g ceftolozane active/500 mg tazobactam active) administered intravenously over one hour every eight hours (see Table 1 below):

TABLE 1
Dosage of CXA-201 by Infection in Patients with Creatinine Clearance
(CrCL) >50 mL/min

| | Dose | | Infusion | Duration | |
|---|---|---|---|---|---|
| | CXA- | | Time | of | |
| Infection | 201 | Frequency | (hours) | Treatment | |
| Complicated Intra-Abdominal | 1.5 g | Every 8 | 1 | 4-14 | days |
| Infections | | Hours | | | |
| Complicated Urinary | 1.5 g | Every 8 | 1 | 7 | days |
| Tract Infections, including | | Hours | | | |
| Pyelonephritis | | | | | |

The intravenous formulation of CXA-201 is prepared by reconstituting a fixed dose combination mixture of two active components (ceftolozane and tazobactam), and intravenously administering the reconstituted pharmaceutical composition. The pharmacokinetic (PK) profile of ceftolozane/tazobactam has been studied in several preclinical and clinical studies. In healthy volunteers the PK of ceftolozane/tazobactam is dose-proportional and linear across a wide range of doses (up to 3000 mg/1500 mg as a single dose) with a terminal elimination half-life (t½β) of approximately 2.5 hours for ceftolozane and 1 hour for tazobactam. Both ceftolozane and tazobactam are primarily excreted in the urine; ceftolozane almost completely in the urine as unchanged parent drug suggesting minimal metabolism, and tazobactam with 80% as the unchanged parent drug and the remaining as inactive M1 metabolite that is formed via hydrolysis of tazobactam (See, e.g., Miller B, Hershberger E, Benziger D, Trinh M, Friedland I., “Pharmacokinetics and safety of intravenous ceftolozane-tazobactam in healthy adult subjects following single and multiple ascending doses,” Antimicrob Agents Chemother. 2012; 56(6):3086-3091).

However, impaired kidney function can result in slower drug clearance of ceftolozane and in increased plasma drug levels. Accordingly, the dosing of CXA-201 in Table 1 is not appropriate for certain patients with advanced renal impairment (including, for example, patients with creatinine clearance less than about 15 mL/minute, such as patients in end stage renal disease who are undergoing hemodialysis). Therefore, there remains a medical need for determining appropriate dosing adjustments for safely and effectively administering a CXA-201 product to a patient at various stages of renal function impairment, including treatment of patients with end stage renal disease (ESRD) (e.g., patients having a creatinine clearance of less than 15 mL/min).

Adjustments in methods of administering other parenteral anti-infective therapies to patients with impaired renal function do not adequately address the unmet medical need for determining safe and effective methods of administering ceftolozane/tazobactam to patients with renal impairment. Modifications in the manner of administering parenteral anti-infective therapies to treat patients with impaired renal function include (1) decreasing the individual dose and increasing the time between doses (e.g., administering 2.25 g of piperacillin/tazobactam every 8 or 12 hours with an additional 0.75 g following dialysis, instead of 3.375 g administered every 6 hours, for treating indicated infections other than nosocomial pneumonia), (2) increasing the time between doses (e.g., administering 1 g of cefepime hydrochloride on day 1 followed by 500 mg every 24 hours thereafter, instead of 0.5-1 or 2 g administered every 12 hours, for urinary tract or intra-abdominal infections), (3) decreasing the amount of individual doses without changing the time between doses (e.g., administering 200 mg of ceftaroline fasamil every 12 hours, instead of 600 mg administered every 12 hours, for certain skin infections or pneumonia), and (4) not changing the dose amount or time between doses (e.g., when administering intravenous linezolid or ceftriaxone sodium to a patient with renal impairment).

Accordingly, there remains a need for safe and effective methods of administering a CXA-201 anti-infective product to patients with impaired renal function, including in patients having ESRD and who are on hemodialysis, to treat complicated urinary tract infections and/or complicated intra-abdominal infections.

### SUMMARY

As disclosed herein, methods for safely and effectively administering a ceftolozane in a pharmaceutical composition (such as a CXA-201 pharmaceutical composition) can include administering a loading dose of ceftolozane followed by a maintenance dose of the ceftolzoane in amounts and at a dosing interval effective to provide a ceftolozane concentration of at least 8 micrograms/mL in the blood for at least 30% of the time between successive doses.

In one preferred embodiment, ceftolozane and tazobactam can be administered in a 2:1 fixed dose combination of ceftolozane active to tazobactam active (“CXA-201”) to a patient having a creatinine clearance of less than 15 mL/minute (e.g., a patient with end stage renal disease and/or on hemodialysis) by parenterally administering to the patient a single loading dose of 750 mg of a CXA-201 pharmaceutical composition (i.e., an amount providing 500 mg of ceftolozane active and 250 mg of tazobactam active), followed by a 150 mg maintenance dose of the CXA-201 pharmaceutical composition (i.e., an amount providing 100 mg of ceftolozane active and 50 mg of tazobactam active) administered every 8 hours for the remainder of the treatment period. The duration of therapy should be guided by the severity and site of infection and the patient's clinical and bacteriological progress (Table 2).

TABLE 2
Dosage of CXA-201 by Infection in ESRD Patients Undergoing Hemodialysis

| | | | Infusion | Duration | |
|---|---|---|---|---|---|
| | | | Time | of | |
| Infection | Dose | Frequency | (hours) | Treatment | |
| Complicated | A single loading dose of 750 mg | Every 8 | 1 | 4-14 | days |
| Intra- | (500 mg ceftolozane active/250 mg | Hours | | | |
| Abdominal | tazobactam active) followed by a | | | | |
| Infections | 150 mg (100 mg ceftolozane active/ | | | | |
| | 50 mg tazobactam active) | | | | |
| | maintenance dose administered | | | | |
| | every 8 hours for the remainder of | | | | |
| | the treatment period (on | | | | |
| | hemodialysis days, the dose should | | | | |
| | be administered at the earliest | | | | |
| | possible time following completion | | | | |
| | of dialysis) | | | | |
| Complicated | A single loading dose of 750 mg | Every 8 | 1 | 7 | days |

| | | |
|---|---|---|
| Urinary Tract | (500 mg ceftolozane active/250 mg | Hours |
| Infections, | tazobactam active) followed by a | |
| including | 150 mg maintenance dose | |
| Pyelonephritis | administered every 8 hours for the | |
| | remainder of the treatment period | |
| | (on hemodialysis days, the dose | |
| | should be administered at the | |
| | earliest possible time following | |
| | completion of dialysis) | |

In one embodiment, the loading dose is administered intravenously over one hour. In another aspect, each maintenance dose is administered intravenously over one hour. In another aspect, the maintenance dose is administered every 8 hours. In another aspect, each maintenance dose is administered intravenously over one hour every eight hours. In yet another aspect, the loading dose is administered intravenously over one hour and each maintenance dose is administered intravenously over one hour every 8 hours.

Typically, the duration of treatment (including loading dose and maintenance dose) for complicated intra-abdominal infections is 4-14 days; and the duration of treatment (including loading dose and maintenance dose) for complicated urinary tract infections is 7 days.

On hemodialysis days, the dose can be administered at the earliest possible time following completion of hemodialysis. For example, the loading dose can be followed immediately after completion of hemodialysis (e.g., within 3 hours, 2 hours, 1 hour, 0.5 hours, 0.25 hours following completion of hemodialysis) by a maintenance dose comprising 100 mg of ceftolozane active and 50 mg tazobactam active. CXA-201 can be administered as a fixed dose ratio of ceftolozane and tazobactam to these patients according to the methods of treatment disclosed herein. These methods of treating ESRD patients undergoing hemodialysis can be used to treat patients having a complicated intra-abdominal infection and/or a complicated urinary tract infection as disclosed herein.

The invention is based in part on the discovery that, following administration of a single 1.5 g intravenous dose of CXA-201 to healthy male adults greater than 95% of ceftolozane was excreted in the urine as unchanged parent drug. More than 80% of tazobactam was excreted as the parent compound with the remainder excreted as the tazobactam M1 metabolite. After a single dose of CXA-201, renal clearance of ceftolozane (3.41-6.69 L/h) was similar to plasma CL (4.10 to 6.73 L/h) and similar to the glomerular filtration rate for the unbound fraction, suggesting that ceftolozane is eliminated by the kidney via glomerular filtration. Approximately 66% of ceftolozane, 56% of tazobactam and 51% of the M1 tazobactam metabolite of tazobactam were removed by dialysis. Furthermore, the inventors discovered the probability of target attainment (PTA) exceeded 90% for a minimum inhibitory concentration (MIC) up to 8 μg/mL for ceftolozane across all the tested scenarios in Example 9. Out of all the tested scenarios, the 500 mg/250 mg C/T single loading dose followed by 100 mg/50 mg every 8 hours maintenance dose via 1-hr infusion achieved a >99% PTA against all targets up to an MIC of 8 μg/mL on day 1 and >97% PTA on all other days without HD. The PTA for bactericidal activity on post HD days was 89%.

### BRIEF DESCRIPTION OF THE DRAWINGS

FIG. 1A shows the demographic and baseline patient characteristics of the mMITT population. No antibiotics were permitted within 48 hours prior to obtaining the baseline urine culture. Urinary catheter was removed before end of treatment in all but three patients in the ceftolozane/tazobactam group and one patient in the levofloxacin group. cLUTI denotes complicated lower urinary tract infections, mMITT microbiological modified intent-to-treat.

FIG. 1B shows the susceptibility of baseline uropathogens to ceftolozane/tazobactam and levofloxacin (mMITT Population). CLSI denotes Clinical and Laboratory Standards Institute, mMITT, microbiologically modified intent-to-treat. Notes: Percentages are calculated as 100×(n/N). If fewer than 10 patients reported an isolate overall, the isolate was reported alongside other isolates of the same genus which meet the same criteria. Not all isolates were received at the central microbiology laboratory for susceptibility testing. For ceftolozane/tazobactam, Susceptible/Indeterminate/Resistant breakpoints were defined as MIC≦8 mg/1; MIC=16 mg/1; and MIC≧32 mg/1, respectively.

FIG. 1C shows the primary and secondary analysis end points at the Test-of-Cure visit (mMITT and per-protocol populations).

FIG. 1D shows the composite cure at the Test-of-Cure visit, according to subgroups (mMITT population). ESBL (extended-spectrum β-lactamase) positive includes isolates of E. coli, K pneumoniae, P. mirabilis, E. cloacae, Enterobacter aerogenes, and Serratia marcescens. cLUTI denotes complicated lower urinary tract infection, mMITT microbiological modified intent-to-treat.

FIG. 1E shows a summary of Adverse Events Occurring in ≧1% of Patients in Either Treatment Group (Safety Population).

FIG. 1F shows a summary of serious adverse events by system organ class (safety population).

FIG. 2 shows summary of 10 clinical studies included in the population-pharmacokinetic analysis. The following abbreviations apply: cIAI, complicated intra-abdominal infections; cUTI, complicated urinary tract infections; ESRD, end-stage renal disease; IV, intravenous; q8 h, every 8 hours; and RI, renal impairment.

FIG. 3 shows the baseline characteristics of subjects included in the population PK model. The following abbreviations apply: BMI, body mass index; CrCL, creatinine clearance; RI, renal impairment; ^{a}includes patients with cUTIs or cIAIs, ^{b}includes patients with cIAIs; ^{c}CrCL ranges for normal, mild, moderate and severe renal impairment were ≧90 mL/min, ≧50-<90 mL/min, ≧30-<50 mL/min and ≧15-<30 mL/min, respectively. CrCL estimated by the Cockcroft-Gault formula.

FIG. 4 shows a Tornado plot showing the effect of infection, renal impairment (based on CrCl categories over a standardized range) and BSV on the relative CL of [A] ceftolozane and [B] tazobactam. Numbers represent the CL range. The following abbreviations apply: BSV, between subject variability; CL, clearance; CrCL, creatinine clearance.

FIG. 5 shows final population-pharmacokinetic models derived for [A] ceftolozane and [B] tazobactam. The following abbreviations apply: BSV, between-subject variability; CL, clearance; CL2, peripheral clearance; CrCl, creatinine clearance; IAI, intra-abdominal infection; NA, not applicable; RSE, relative standard error; UTI, urinary tract infection; Vc, central volume of distribution; Vp, peripheral volume of distribution.

FIG. 6 shows population and individual predicted versus observed plasma concentrations of [A] ceftolozane and [B] tazobactam for the final PK model (Goodness of fit plot). The following abbreviations apply: CWRES: Conditional weighted residuals; DV: Observed concentration; IDENT: Identity line; IPRED: Individual predicted concentrations; LOESS: Locally weighted scatter smoothing; PK: Pharmacokinetic; PRED: Population predicted concentrations; TAD: Time after last dose.

FIG. 7 shows two graphs of the simulated probability of target attainment on day 1 (top, with loading dose) and day 3 (bottom, post HD) in patients with ESRD on HD (n=5000) after administration of a loading dose of 500 mg/250 mg C/T, followed in 8 hr by 100 mg/50 mg C/T, 1-hr IV infusion every 8 hours.

FIG. 8 shows the baseline characteristics of subjects. ^{a} RI, renal impairment. ^{b}CrCl estimated by the Cockcroft-Gault formula. ^{c}BMI, body mass index. ^{d}NA, not applicable.

FIG. 9 shows the median pharmacokinetic values for ceftolozane following single-dose administration of intravenous ceftolozane/tazobactam. ^{a} C/T, ceftolozane/tazobactam. ^{b}The t_{1/2} on HD was calculated from the terminal elimination phase post HD. ^{c}Incomplete urine recovery over 48 h. ^{d}ND, not determined. As a majority of the subjects with ESRD were anuric, CL, could not be determined.

FIG. 10 shows the median pharmacokinetic values for tazobactam following single-dose administration of intravenous ceftolozane/tazobactam. ^{a} The t_{1/2} on HD was calculated from the terminal elimination phase post HD.

FIG. 11 shows the median (range) plasma concentration-time profiles of [A] ceftolozane and [B] tazobactam following single-dose administration of intravenous ceftolozane/tazobactam (semi-log plot). ^{a}C/T, ceftolozane/tazobactam. ^{b}RI, renal impairment.

FIG. 12 shows regression plots of [A] ceftolozane and [B] tazobactam plasma clearance versus CrCl following single-dose administration of intravenous ceftolozane/tazobactam. ^{a}C/T, ceftolozane/tazobactam. ^{b}RI, renal impairment.

FIG. 13 shows the median (range) plasma concentration-time profiles of ceftolozane and tazobactam following administration of intravenous ceftolozane/tazobactam (500 mg/250 mg) in subjects with ESRD on [A] day 1 (post-HD) and [B] day 4 (on HD) (semi-log plot).

FIG. 14 shows the characteristics of the dialysis criteria in the safety population. Unless otherwise indicated herein, clinical data disclosed herein obtained in patients on hemodialysis (HD) with end stage renal disease (ESRD) subjects, including BUN collected before and after dialysis, was obtained using HD dialysis parameters provided in FIG. 14. Note: A=Asian; B=Black or African American; P=Native Hawaiian or Other Pacific islander; I=American Indian or Alaska Native; 0=Other; W=White; H=Hispanic or Latino; N=Not Hispanic or Latino; M=Male; F=Female. Unless otherwise indicated herein, subjects underwent HD for 3 to 4 hours using a high-flux membrane (either 1.4, 1.8, or 1.9 m2) on Days 1, 4, and 6 as scheduled. Average blood flow rate was 264 to 600 mL/min and average dialysis flow rate was either 600 or 800 mL/min for all subjects with ESRD.

### DETAILED DESCRIPTION

In one aspect, the instant disclosure provides a method of treating a complicated intra-abdominal infection or a complicated urinary tract infection in a human patient having a creatinine clearance of less than 15 mL/minute, the method comprising intravenously administering to the patient 500 mg of ceftolozane active and 250 mg tazobactam active, followed by administering one or more additional doses of 100 mg ceftolozane active and 50 mg of tazobactam active to the patient every 8 hours for the duration of a treatment period.

In another aspect, the instant disclosure provides a method of treating a complicated intra-abdominal infection or a complicated urinary tract infection in a human patient with end stage renal disease with a ceftolozane/tazobactam composition that includes ceftolozane or a pharmaceutically acceptable salt thereof combined in a fixed dose ratio with tazobactam or a pharmaceutically acceptable salt thereof in an amount providing a 2:1 weight ratio between the amount of ceftolozane active and the amount of tazobactam active in the ceftolozane/tazobactam composition, where the method comprises intravenously administering to the human patient a single loading dose comprising 750 mg of the ceftolozane/tazobactam composition followed by a maintenance dose of the antibiotic composition comprising 150 mg of the ceftolozane/tazobactam composition.

In another aspect, the instant disclosure provides a method of treating a complicated intra-abdominal infection or a complicated urinary tract infection in a human patient having a creatinine clearance of less than 15 mL/minute, the method comprising intravenously administering to the patient a first pharmaceutical composition comprising 500 mg of ceftolozane active and 250 mg tazobactam active, followed by administering one or more additional doses of a second pharmaceutical composition comprising 100 mg ceftolozane active and 50 mg of tazobactam active to the patient every 8 hours for the duration of a treatment period.

In another aspect, the instant disclosure provides a method of treating a complicated intra-abdominal infection or a complicated urinary tract infection in a human patient having a creatinine clearance of less than 15 mL/minute, the method comprising intravenously administering to the patient a first liquid pharmaceutical composition comprising 500 mg of a compound of formula (I)

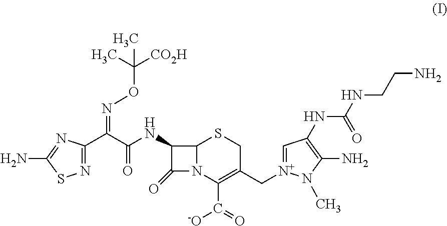

and 250 mg of a compound of formula (II)

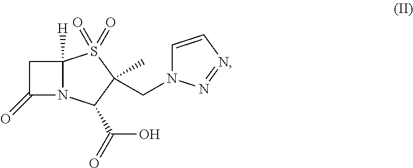 [SMILES: C[C@]1(Cn2ccnn2)[C@H](C(=O)O)N2C(=O)C[C@H]2S1(=O)=O]

followed by administering one or more additional doses of a second liquid pharmaceutical composition comprising 100 mg the compound of formula (I) and 50 mg of the compound of formula (II) to the patient every 8 hours for the duration of a treatment period.

Ceftolozane (formula (I) below) is a cephalosporin antibacterial agent, also referred to as CXA-101, FR264205, or by chemical names such as (6R,7R)-3-[(5-amino-4-{[(2-aminoethyl)carbamoyl]amino}-1-methyl-1H-pyrazol-2-ium-2-yl)methyl]-7-({(2Z)-2-(5-amino-1,2,4-thiadiazol-3-yl)-2-[(1-carboxy-1-methylethoxy)imino]acetyl}amino)-8-oxo-5-thia-1-azabicyclo[4.2.0]oct-2-ene-2-carboxylate, and 7β-[(Z)-2-(5-amino-1,2,4-thiadiazol-3-yl)-2-(1-carboxy-1-methylethoxyimino)acetamido]-3-{3-amino-4-[3-(2-aminoethyl)ureido]-2-methyl-1-pyrazolio}methyl-3-cephem-4-carboxylate. The antibacterial activity of ceftolozane is believed to result from its interaction with penicillin binding proteins (PBPs) to inhibit the biosynthesis of the bacterial cell wall which acts to stop bacterial replication.

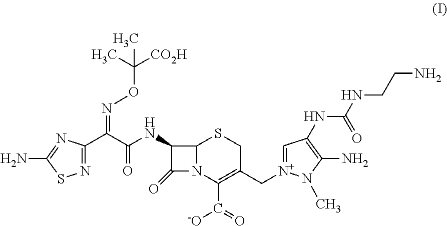

Unless otherwise indicated herein, the term “ceftolozane” refers to pharmaceutically acceptable salts of ceftolozane as well as ceftlolozane free base. Ceftolozane sulfate is an example of a pharmaceutically acceptable ceftolozane salt of the compound of formula (I) that can be formulated for intravenous administration or infusion. As used herein, unless otherwise indicated, the term “ceftolozane active” refers to the active portion of a salt form of ceftolozane (e.g., ceftolozane sulfate), i.e., the free base form of ceftolozane. As used herein, unless otherwise indicated, the phrase “1,000 mg of ceftolozane as ceftolozane active” refers to an amount of ceftolozane in any form, including a ceftolozane salt (e.g., ceftolozane sulfate), in an amount that provides 1,000 mg of the ceftolozane active moiety. As used herein, “TOL” can refer to ceftolozane or a pharmaceutically acceptable salt thereof.

Ceftolozane can be combined with the β-lactamase inhibitor (“BLI”) tazobactam to form an antibiotic pharmaceutical composition suitable for intravenous administration. Tazobactam is a BLI against Class A and some Class C β-lactamases, with in vitro and in vivo efficacy in combination with active beta-lactam antibiotics. The term “tazobactam” refers to the free acid tazobactam form of formula (II), as well as pharmaceutically acceptable salts of the compound of formula (II)).

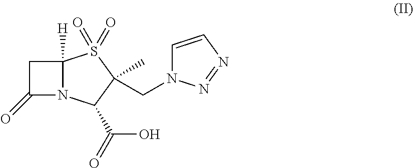 [SMILES: C[C@]1(Cn2ccnn2)[C@H](C(=O)O)N2C(=O)C[C@H]2S1(=O)=O]

Commonly used tazobactam salts include the sodium salt or arginine salt. Tazobactam can also be a hydrate or solvate. “Tazobactam active” refers to the active portion of a salt form of tazobactam, i.e., tazobactam free acid. For example, “500 mg of tazobactam as tazobactam active” refers to an amount of the salt form of tazobactam (e.g., tazobactam sodium or tazobactam arginine) effective to provide 500 mg of tazobactam active. As used herein, “TAZ” can refer to tazobactam or a pharmaceutically acceptable salt thereof.

A CXA-201 composition includes ceftolozane (e.g., as a pharmaceutically acceptable ceftolozane salt) and tazobactam (e.g., as a pharmaceutically acceptable tazobactam salt) in amounts that provide a 2:1 ratio between the amount of ceftolozane active and the amount of the tazobactam active. A preferred antibiotic composition used in the disclosed methods contains 2:1 w/w of ceftolozane active/tazobactam active formulated for parenteral administration. In one aspect, the antibiotic composition is referred to as “CXA-201” and comprises ceftolozane sulfate and tazobactam sodium in sufficient quantities to provide 2:1 w/w of ceftolozane active/tazobactam active. In another aspect, CXA-201 is formulated for parenteral administration. As used herein, “TOL/TAZ” refers to a CXA-201 composition.

CXA-201 can be provided as a lyophilized powder of ceftolozane sulfate and tazobactam sodium ready for reconstitution. In one aspect, the unit dosage form of CXA-201 is provided in a vial ready for reconstitution. The pharmaceutical composition can be obtained by combining the ceftolozane composition with a (second) tazobactam composition (e.g., preferably, but not necessarily, prepared in the absence of ceftolozane) by forming a second solution comprising tazobactam. The tazobactam can be included in an amount providing about 5 mg of tazobactam active per 10 mg ceftolozane active (i.e., a 1:2 weight ratio of tazobactam active to ceftolozane active). Tazobactam is a beta-lactamase inhibitor in its free acid form. Unless otherwise indicated, tazobactam can be a free acid, a sodium salt, an arginine salt, or a hydrate or solvate thereof. In one embodiment, the tazobactam in the (second) tazobactam composition is tazobactam acid and the second composition further comprises sodium bicarbonate or sodium hydroxide. Lyophilizing tazobactam in the presence of sodium bicarbonate or sodium hydroxide forms a lyophilized tazobactam sodium, which can then be further blended with the (first) lyophilized ceftolozane composition.

Pharmaceutical compositions can be obtained by lyophilization. Specific methods of lyophilization are described in Remington's Pharmaceutical Sciences, Chapter 84, page 1565, Eighteenth Edition, A. R. Gennaro, (Mack Publishing Co., Easton, Pa., 1990).

Pharmaceutical compositions comprising ceftolozane and tazobactam can be formulated to treat infections by parenteral administration (including subcutaneous, intramuscular, and intravenous) administration. Pharmaceutical compositions may additionally comprise excipients, stabilizers, pH adjusting additives (e.g., buffers) and the like. Non-limiting examples of these additives include sodium chloride, citric acid and L-arginine. For example, the use of sodium chloride results in greater stability; L-arginine is used to adjust pH and to increase the solubility of ceftolozane; and citric acid is used to prevent discoloration of the product, due to its ability to chelate metal ions. In one particular embodiment, the pharmaceutical compositions described herein are formulated for administration by intravenous injection or infusion.

The pharmaceutical antibiotic compositions can be provided in a unit dosage form container (e.g., in a vial or bag, or the like). The unit dosage form can be dissolved with a pharmaceutically acceptable carrier, and then intravenously administered. The unit dosage form comprises ceftolozane active and tazobactam active, typically 1000 mg ceftolozane active as ceftolozane sulfate and 500 mg of tazobactam active as tazobactam sodium. The unit dosage forms are commonly stored in vials.

In one aspect, provided herein is a unit dosage form container (e.g., a bag, vial or the like) containing a unit dosage form of a pharmaceutical composition formulated for parenteral administration for the treatment of complicated intra-abdominal infections, the pharmaceutical composition comprising a therapeutically effective amount of ceftolozane sulfate and tazobactam in a ratio of 1,000 mg ceftolozane active per 500 mg of tazobactam active, the pharmaceutical composition obtained by a process comprising the steps of:

a. lyophilizing a first aqueous solution in the absence of tazobactam, the first aqueous solution comprising ceftolozane sulfate, 125 mg to 500 mg of sodium chloride per 1,000 mg of ceftolozane active, L-arginine and/or citric acid in an amount effective to adjust the pH of the first aqueous solution to 5-7 (e.g., 6-7) prior to lyophilization to obtain a first lyophilized ceftolozane composition,

b. lyophilizing a second solution in the absence of ceftolozane, the second solution comprising tazobactam being lyophilized to form a second lyophilized tazobactam composition; and

c. blending the first lyophilized ceftolozane composition and the second lyophilized tazobactam composition to obtain a blended pharmaceutical composition in the unit dosage form.

In one embodiment of the unit dosage form container, the tazobactam in the second solution is tazobactam acid, and wherein the tazobactam acid in the second solution is lyophilized in the presence of sodium bicarbonate or sodium hydroxide, thereby forming lyophilized tazobactam sodium in the second lyophilized tazobactam solution.

The pharmaceutical compositions provided herein comprising ceftolozane sulfate and tazobactam in a ratio of 1,000 mg ceftolozane active per 500 mg of tazobactam active, can be obtained by a process comprising the steps of:

a. lyophilizing a first aqueous solution in the absence of tazobactam, the first aqueous solution comprising ceftolozane sulfate at a pH of 5-7 (e.g, 6-7) prior to lyophilization to obtain a first lyophilized ceftolozane composition,

b. blending the first lyophilized ceftolozane composition with tazobactam to obtain an antibacterial composition.

As provided herein, ceftolozane can be stabilized in a pharmaceutical composition comprising ceftolozane and a stabilizing effective amount of a stabilizing agent selected from the group consisting of: sodium chloride, dextran 40, lactose, maltose, trehalose and sucrose. The pharmaceutical compositions provided herein are based in part on the surprising discovery that ceftolozane pharmaceutical compositions comprising these stabilizing agents demonstrate improved ceftolozane residual rates (e.g., % ceftolozane remaining after 3 days at 70 degrees C. as measured by HPLC) and/or chemical stability (e.g., lower reduction in ceftolozane purity measured by HPLC after 7 days at 60 degrees C. in a stability test) compared control samples comprising ceftolozane without a stabilizing agent.

Accordingly, preferred pharmaceutical antibiotic compositions can include ceftolozane sulfate and a stabilizing agent (e.g., 300 to 500 mg of a stabilizing agent per 1,000 mg ceftolozane active) in a lyophilized unit dosage form (e.g., powder in a container). The unit dosage form can be dissolved with a pharmaceutically acceptable carrier (e.g., 0.9% sodium chloride aqueous isotonic saline and/or water for injection), and then intravenously administered. In certain ceftolozane compositions, the stabilizing agent can be selected from the group consisting of: sodium chloride, lactose, maltose and dextran 40, and/or selected from the group consisting of: sodium chloride, trehalose and sucrose.

An exemplary unit dosage form is described in Example 3, a white to yellow sterile powder consisting of ceftolozane sulfate (1147 mg/vial) and tazobactam sodium (537 mg/vial) packaged in glass vials. The product contains sodium chloride (487 mg/vial) as a stabilizing agent, citric acid (21 mg/vial), and L-arginine (approximately 600 g/vial) as excipients. The vial with the unit dosage form is constituted with 10 mL of sterile water for injection or 0.9% Sodium Chloride for injection, USP (normal saline) and gently shaken to dissolve. In another aspect, 10 mL of 5% Dextrose Injection, USP is used. The final volume is approximately 11.4 mL. The resultant concentration is approximately 132 mg/mL. For preparation of a 1.5 g dose, the entire contents (approximately 11.4 mL) of the reconstituted vial is removed, for example, by using a syringe, and added to an infusion bag containing 100 mL of 0.9% Sodium Chloride for Injection, USP (normal saline) or 5% Dextrose Injection, USP. In another aspect, 100 mL of sterile water for injection can be used. For preparation of the 750 mg dose, approximately 5.7 mL of the contents of the reconstituted vial is withdrawn and added it to an infusion bag containing 100 mL of 0.9% Sodium Chloride for Injection, USP (normal saline) or 5% Dextrose Injection, USP. In another aspect, 100 mL of sterile water for injection is used. For preparation of the 375 mg dose, approximately 2.9 mL of the contents of the reconstituted vial is withdrawn and added to an infusion bag containing 100 mL of 0.9% Sodium Chloride for Injection, USP (normal saline) or 5% Dextrose Injection, USP. In another aspect, 100 mL of sterile water for injection is used. For preparation of the 150 mg dose, approximately 1.4 mL of the contents of the reconstituted vial is withdrawn and added it to an infusion bag containing 100 mL of 0.9% Sodium Chloride for Injection, USP (normal saline) or 5% Dextrose Injection, USP. In another aspect, 100 mL of sterile water for injection is used.

Preferably, the ceftolozane/tazobactam pharmaceutical product does not contain a bacteriostatic preservative. Aseptic technique is preferably followed in preparing the infusion solution.

A therapeutically effective amount of metronidazole or a pharmaceutically acceptable salt thereof can be administered to a patient receiving the ceftolozane/tazobactam pharmaceutical composition for treatment of an intra-abdominal infection, including a complicated intra-abdominal infection. Metronidazole is a synthetic nitroimidazole antibacterial agent 2-methyl-5-nitro-1Himidazole-1-ethanol. Metronidazole hydrochloride (formula III) is a pharmaceutically acceptable salt of metronidazole that can be intravenously administered.

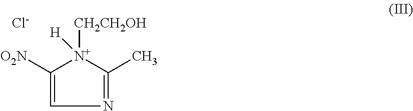

An effective amount of metronidazole is administered in the treatment methods described herein. Metronidazole is preferably administered intravenously using a dosage regimen of 15 mg/kg loading dose (1 gram for a 70 kg adult) followed six hours later by 7.5 mg/kg (500 mg for a 70 kg adult) maintenance dose. Maintenance doses of 7.5 mg/kg are given intravenously every six hours. The usual duration of therapy for treating the intra-abdominal infection can be 4-14 days, possibly 7-10 days. Metronidazole is preferably administered separately from the ceftolozone/tazobactam.

Preferably, the metronidazole is separately intravenously administered to a patient having, for example, an intra-abdominal infection. Metranodazole is an antibiotic that can be administered to patients having intra-abdominal infections, including peritonitis, intra-abdominal abscess, and liver abscess, caused by Bacteroides species including the B. fragilis group (B. fragilis, B. distasonis, B. ovatus, B. thetaiotaomicron, B. vulgatus), Clostridium species, Eubacterium species, Peptococcus species, and Peptostreptococcus species.

Preferably, the metronidazole is intravenously administered as a pharmaceutically composition of metronidazole hydrochloride for injection in a sterile 500 mg parenteral unit dosage form of the synthetic nitroimidazole antibacterial agent 2-methyl-5-nitro-1Himidazole-1-ethanol. The unit dosage form of metronidazole can be obtained by reconstituting a single-dose vial of lyophilized metronidazole hydrochloride (e.g., sold under the brand name FLAGYL I.V.) containing sterile, nonpyrogenic metronidazole hydrochloride, equivalent to 500 mg metronidazole, and 415 mg mannitol.

CXA-201 is useful for the treatment of complicated intra-abdominal infections caused by one of the following Gram-negative and Gram-positive microorganisms: Citrobacter freundii, Enterobacter cloacae, Escherichia coli, Escherichia coli CTX-M-14 extended spectrum beta-lactamase producing strains, Escherichia coli CTX-M-15 extended spectrum beta-lactamase producing strains, Klebsiella oxytoca, Klebsiella pneumonia, Klebsiella pneumoniae CTX-M-15 extended spectrum beta-lactamase producing strains, Proteus mirabilis, Pseudomonas aeruginosa, Bacteroides fragilis, Bacteroides ovatus, Bacteroides thetaiotaomicron, Bacteroides vulgatus, Streptococcus anginosus, Streptococcus constellatus, and Streptococcus salivarius.

CXA-201 is also useful for the treatment of complicated urinary tract infection, including where the complicated urinary tract infection is pyelonephritis, with or without concurrent bacteremia, or is caused by one of the following Gram-negative microorganisms: Escherichia coli, Escherichia coli levofloxacin resistant strains, Escherichia coli CTX-M-14 extended spectrum beta-lactamase producing strains, Escherichia coli CTX-M-15 extended spectrum beta-lactamase producing strains, Klebsiella pneumoniae, Klebsiella pneumonia levofloxacin resistant strains, Klebsiella pneumonia CTX-M-15 extended spectrum beta-lactamase producing strains, Proteus mirabilis or Pseudomonas aeruginosa.

CXA-201 can also be used to treat infections caused by the following bacteria: Gram-negative bacteria—Acinetobacter baumannii, Burkholderia cepacii, Citrobacter freundii, Citrobacter koseri, Enterobacter aerogenes, Enterobacter cloacae, Haemophilus influenza, Moraxella catarrhalis, Morganella morganii, Pantoea agglomerans, Proteus vulgaris, Providencia rettgeri, Providencia stuartii, Serratia liquefacians and Serratia marcescens; Gram-positive aerobic bacteria—Streptococcus agalactiae, Streptococcus intermedius, Streptococcus pyogenes and Streptococcus pneumonia; Anaerobic microorganisms such as Fusobacterium spp, and Prevotella spp.

The invention is illustrated by the following non-limiting examples.

### EXAMPLES

### Example 1

### Manufacturing Procedure of Bulk (Tray) Lyophilized Ceftolozane

There are four main steps in the manufacture of ceftolozane bulk drug product: dissolution, sterile filtration, bulk lyophilization, and packaging into Sterbags®. These four main steps are composed of a total of 20 minor steps. The ceftolozane bulk drug product manufacturing process is presented below.

### Dissolution

The prescribed amount of water for injection (“WFI”) is charged into the dissolution reactor.
A prescribed amount of citric acid is added.
The solution is cooled at 5° C. to 10° C.
A prescribed amount of ceftolozane drug substance is added to the solution.
A prescribed amount of L-arginine is slowly added to the solution.
A check for complete dissolution is performed. Solution pH is verified to be in the target range of 6.5 to 7.0.
A prescribed amount of sodium chloride is added to the solution.
A check for complete dissolution is performed. Solution pH is verified to be in the target range of 6.0 to 7.0. If the pH is out of this range adjust with either L-Arginine or citric acid.
WFI is added to bring the net weight to 124.4 kg and the solution is mixed well.
Samples are withdrawn for testing of final pH.
   Sterile filtration
The solution is passed through the filter (pore size 0.45 μm) followed by double filters (pore size 0.22 μm) onto a shelf on the Criofarma lyophilizer.
The line is washed with WFI.
The washing solution is passed from Step 12 through sterile filtration.
Bulk lyophilization
The washing solution is loaded onto a separate shelf in the lyophilizer (and later discarded).
The solution is lyophilized until dry.
The product shelf is cooled to 20° C.±5° C.
   Packaging into Sterbags®
The lyophilized bulk drug product powder is milled.
The milled powder is sieved.
The sieved powder is blended for 30 minutes.
The powder is then discharged into Sterbags®

### Prefiltration and Sterile-Filtration

Filter the compounded solution with a sterile tilter-set which consists of a 0.2 um polyvinylidene fluoride membrane filter (Durapore®, Millipore) and a 0.1 urn polyvinylidene fluoride membrane filter (Durapore®, Millipore) connected in tandem. Confirm the integrity of each filter before and after the filtration. Take approximately 100 mL of the filtrate in order to check bioburden.
Filter the prefiltered compounded solution through a sterile filter-set which consists of a 0.2 um polyvinylidene fluoride membrane filter and a 0.1 urn polyvinylidene fluoride membrane filter connected in tandem, and introduce the final filtrate into an aseptic room. Confirm the integrity of each filter before and after the filtration.

### Processing of Vial, Stopper and Flip-Off Cap

Wash a sufficient quantity of 28 mL vials with water for injection and sterilize the washed vials by a dry-heat sterilizer. Then transfer the sterilized vials into a Grade A area located in an aseptic room.
Wash a sufficient quantity of stoppers with, water for injection. Sterilize and dry the washed stoppers by steam sterilizer. Then transfer the sterilized stoppers into a Grade A area located in an aseptic room.
Sterilize a sufficient quantity of flip-off caps by steam sterilizer. Then transfer the sterilized flip-off caps into a Grade A or B area located in an aseptic room.

### Filling and Partially Stoppering

Adjust the fill weight of the filtered compounded solution to 11.37 g (corresponds to 10 mL of the compounded solution), then start filling operation. Check the filled weight in sufficient frequency and confirm it is in target range (11.37 g±1%, 11.26 to 11.43 g). When deviation from the control range (11.37 g±2%, 11.14 to 11.59 g) is occurred, re-adjust the filling weight.
Immediately after a vial is filled, partially stopper the vial with a sterilized stopper. Load the filled and partially stoppered vials onto the shelves of a lyophilizer aseptically. Lyophilization to Crimping, Visual Inspection, Labeling and Packaging

After all filled and partially stoppered vials are loaded into a lyophilizer, start the lyophilization program. Freeze the loaded vials at −40° C. and keep until all vials freeze. Forward the program to primary drying step (shelf temperature; −20° C., chamber pressure; 100 to 150 mTorr). Primary drying time should be determined by monitoring the product temperature. Forward the program to secondary drying step (shelf temperature; 30° C., chamber pressure; not more than 10 mTorr) after completion of the primary drying step. After all vials are dried completely, return the chamber pressure to atmospheric pressure with sterilized nitrogen. Then stopper vials completely. Unload the lyophilized vials from the chamber and crimp with sterilized flip-off caps. Subject all crimped vials to visual inspection and label and package all passed vials.

### Example 2

### Manufacturing of Combination Product (Tazobactam and Ceftolozane) by Blending

The ceftolozane produced by Example 1 was blended with lyophilized tazobactam, to provide a ceftolozane composition. A low energy drum blender that agitates the material by tumbling and also moving the bed up and down is used. For ceftolozane/tazobactam for injection, the blender was charged with 23.4 kg of ceftolozane bulk product, and 5.4 kg of tazobactam bulk product. Both the ceftolozane and tazobactam were individually lyophilized beforehand. The material was blended for 180 minutes. In-process tests of content assay for both ceftolozane and tazobactam were performed to assess the homogeneity using the samples of blend materials taken from three places. The RSD for each of ceftolozane and tazobactam content assay was no greater than 2% and the RSD for the ratio of ceftolozane/tazobactam was no greater than 2.2%.

### Example 3

### Components of a Representative CXA-201 Composition

An example of a batch formulae for ceftolozane composition (compounding of ceftolozane substance with excipients such as citric acid, sodium chloride, and L-arginine followed by sterile lyophilization) is found below in Table 3.

TABLE 3
Batch Formula for Ceftolozane Composition

| | Target | Amount per | |
|---|---|---|---|
| | Composition | Batch (kg) | |
| Component | mg/g | 1 | 2 |
| Ceftolozane Sulfate¹⁾ | 172.1 | 114.0 | 202.6 |
| Citric Acid, Anhydrous, | 3.2 | 2.1 | 3.7 |
| USP | | | |
| Sodium Chloride, USP | 73.1 | 48.3 | 86.0 |
| L-Arginine, USP | ~90 | 59.7 | 106.0 |
| | QS to achieve target | | |
| | pH²⁾ | | |
| Water for Injection, USP | QS to 1000 | QS | QS |

| | | |
|---|---|---|
| Total Batch Size | 660 | 1175 |

¹⁾Ceftolozane sulfate is charged based on its measured potency to obtain 150 mg free base/g solution.
²⁾L-arginine is added as needed to obtain pH 6.5 ± 0.5 in the bulk solution; 90 mg per gram solution is considered a representative amount.

An example of a batch formula for the ceftolozane/tazobactam drug product is presented in Table 4 below.

TABLE 4
Batch Formula Ceftolozane/Tazobactam Drug Product

| | Amount per | Amount per |
|---|---|---|
| Component | container, mg | Batch, kg |
| Ceftolozane | 2255 | 112.8 |
| composition1) | | |
| Tazobactam2) | 537 | 26.9 |
| Nitrogen, NF3) | — | — |
| Total | 2792 | 139.7 |

| | |
|---|---|
| Total Batch Size, kg | 139.7 |
| Total container Quantity | 50,000 |

The target fill for ceftolozane is 1000 mg free base, added to the container as the composition. The amount 2255 mg is based on 100% theoretical potency of the composition. Actual weight will vary based on composition measured potency. The target fill for tazobactam is 500 mg free acid, added to the container as its sodium salt form. The amount 537 mg is based on 100% theoretical potency. Nitrogen is used as a processing aid to blanket containers after powder filling and prior to insertion of stopper. The unit composition of a dosage for reconstitution is described in Table 5.

TABLE 5
Unit Compositions of Ceftolozane/Tazobactam
for Injection, 1000 mg/500 mg

| | | | Nominal Composition |
|---|---|---|---|
| Component | | Function | mg per container |
| Ceftolozane | Ceftolozane | Active | 1147 |
| composition¹⁾ | Sulfate | | |
| | Citric Acid, | Chelating Agent | 21 |
| | Anhydrous | | |
| | Sodium | Stabilizing Agent | 487 |
| | Chloride | | |
| | L-Arginine | Alkalizing Agent | 600²⁾ |
| | | | Q.S. for pH adjustment |

| | | |
|---|---|---|
| Tazobactam Sodium³⁾ | Active | 537 |
| Nitrogen | Processing Aid^{(a)} | Q.S. |
| Total Weight | | 2792 |

¹⁾Actual amount of ceftolozane composition will vary based on the measured potency. Ceftolozane sulfate, 1147 mg, corresponds to 1000 mg ceftolozane free base.
²⁾L-arginine is added as needed to achieve pH 6.5 ± 0.5; 600 mg per container is considered a representative total amount.
³⁾Actual weight of tazobactam sodium will vary based on the measured potency. Tazobactam sodium 537 mg, corresponds to 500 mg tazobactam free acid.
⁴⁾Nitrogen blanket is applied after powders are dispensed to the container and prior to insertion of stopper.

### Example 4

### Clinical Trials of CXA-201 in Patients with Complicated Intra-Abdominal Infections

A total of 979 adults hospitalized with complicated intra-abdominal infections were randomized and received study medications in a multinational, double-blind study comparing CXA-201 (1.5 g IV every 8 hours) plus metronidazole (500 mg IV every 8 hours) to meropenem (1 g IV every 8 hours) for 4 to 14 days of therapy. Complicated intra-abdominal infections included appendicitis, cholecystitis, diverticulitis, gastric/duodenal perforation, perforation of the intestine, and other causes of intra-abdominal abscesses and peritonitis. The primary efficacy endpoint was clinical response at the test-of-cure (TOC) visit in the microbiological intent-to-treat (MITT) population, which included all patients who had at least 1 baseline intra-abdominal pathogen. The key secondary efficacy endpoint was clinical response at the TOC visit in the microbiologically evaluable (ME) population, which included all protocol-adherent MITT patients.

The MITT population consisted of 806 patients; the median age was 52 years and 57.8% were male. Diffuse peritonitis at baseline, a marker of severity, was present in 34.2% of patients. Laparotomy was the initial intra-abdominal intervention in 67.7% of patients, and ESBL-producing Enterobacteriaceae were identified in 58 (7.2%) patients at baseline.

CXA-201 plus metronidazole showed non-inferiority to meropenem with regard to clinical cure rates at the TOC visit in both the MITT and ME populations. Clinical cure rates at the TOC visit are displayed by patient population in Table 6. Clinical cure rates at the TOC visit by pathogen in the ME population are presented in Table 7.

TABLE 6
Clinical Cure Rates in a Phase 3 Study of Complicated Intra-Abdominal
Infections

| | CXA-201 plus | | Percentage |
|---|---|---|---|
| Analysis | metronidazole^{a} | Meropenem^{b} | Difference |
| Population | n/N (%) | n/N (%) | (95% CI)^{c} |
| MITT | 323/389 (83.0) | 364/417 (87.3) | −4.2 (−8.91, 0.54) |
| ME | 259/275 (94.2) | 304/321 (94.7) | −1.0 (−4.52, 2.59) |

^{a}CXA-201 1.5 g IV every 8 hours + metronidazole 500 mg IV every 8 hours
^{b}1 g IV every 8 hours.
^{c}The 95% CI was calculated using the Newcombe method with minimum risk weights

TABLE 7
Per Pathogen Clinical Cure Ratesin a Phase 3 Study of Complicated Intra-
abdominal Infections (ME Population)

| | CXA-201 plus | |
|---|---|---|
| Pathogen Category | metronidazole | Meropenem |
| Baseline Intra-abdominal Pathogen | n/N (%) | n/N (%) |
| Aerobic Gram-negative | 238/252 (94.4) | 273/291 (93.8) |
| Escherichia coli | 197/208 (94.7) | 216/231 (93.5) |
| Escherichia coli (EBSL-producing) | 14/14 (100) | 18/20 (90.0) |
| Escherichia coli (CTX-M-14/15 | 9/9 (100) | 7/9 (77.8) |
| ESBL-producing) | | |
| Klebsiella pneumonia | 28/30 (93.3) | 22/25 (88.0) |
| Klebsiella pneumoniae (ESBL- | 7/8 (87.5) | 3/4 (75.0) |
| producing) | | |
| Klebsiella pneumoniae (CTX-M-15 | 9/9 (100) | 7/9 (77.8) |
| ESBL-producing) | | |
| Pseudomonas aeruginosa | 26/26 (100) | 27/29 (93.1) |
| Enterobacter cloacae | 19/22 (86.4) | 22/22 (100) |
| Klebsiella oxytoca | 12/12 (100) | 21/22 (95.5) |
| Proteus mirabilis | 10/11 (90.9) | 9/10 (90.0) |
| Aerobic Gram-positive | 153/168 (91.1) | 170/185 (91.9) |
| Streptococcus anginosus | 25/30 (83.3) | 23/23 (100) |
| Streptococcus constellatus | 17/18 (94.4) | 20/23 (87.0) |
| Streptococcus salivarius | 9/10 (90.0) | 8/8 (100) |
| Anaerobic Gram-negative | 104/109 (95.4) | 132/137 (96.4) |
| Bacteroides fragilis | 39/41 (95.1) | 56/57 (98.2) |
| Bacteroides ovatus | 36/37 (97.3) | 42/42 (100) |
| Bacteroides thetaiotaomicron | 20/20 (100) | 40/43 (93.0) |
| Bacteroides vulgatus | 12/13 (92.3) | 21/22 (95.5) |

### Example 5

### Clinical Trials with CXA-201 in Patients with Complicated Urinary Tract Infections, Including Pyelonephritis

A total of 1068 adults hospitalized with complicated urinary tract infections (including pyelonephritis) were randomized and received study medications in a multinational, double-blind study comparing CXA-201 (1.5 g IV every 8 hours) to levofloxacin (750 mg IV once daily) for 7 days of therapy. The primary efficacy endpoint was the composite microbiological and clinical cure response at the test-of-cure (TOC) visit in the microbiologically modified intent-to-treat (mMITT) population, which included all patients who received study medication and had at least 1 baseline uropathogen. The key secondary efficacy endpoint was the composite microbiological and clinical cure response at the TOC visit in the microbiologically evaluable (ME) population, which included protocol-adherent mMITT patients with a urine culture at the TOC visit.

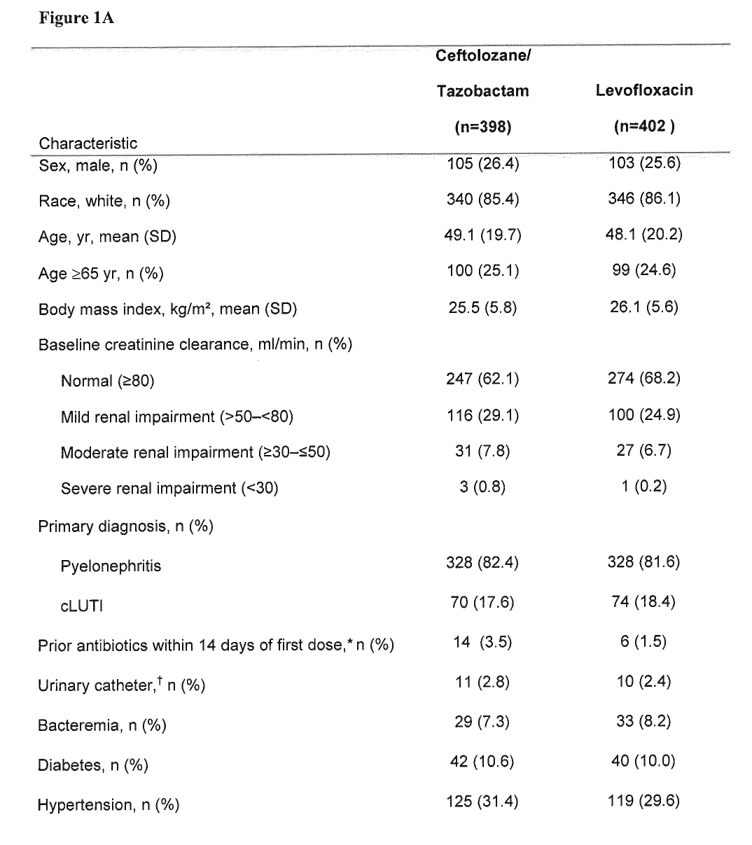

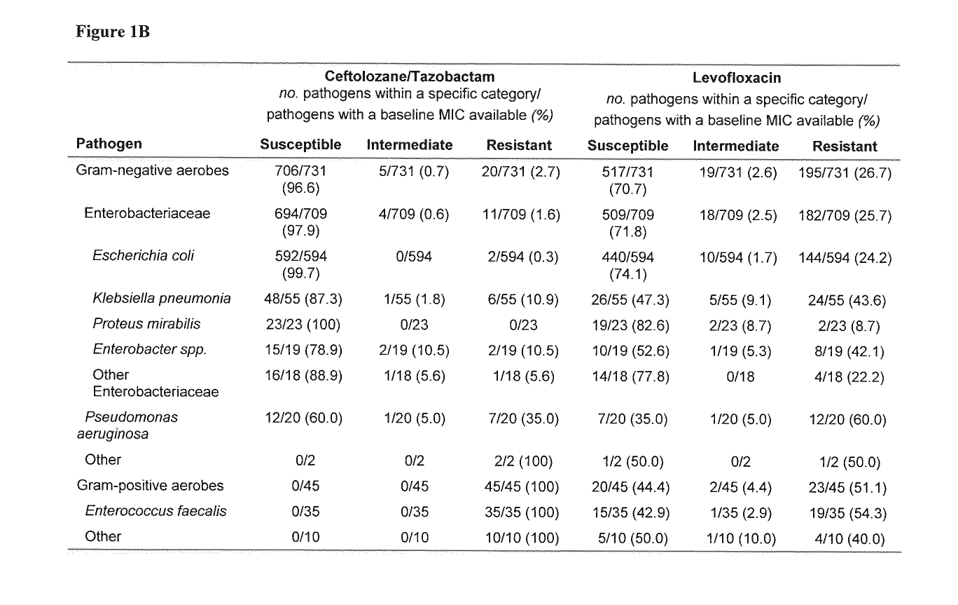

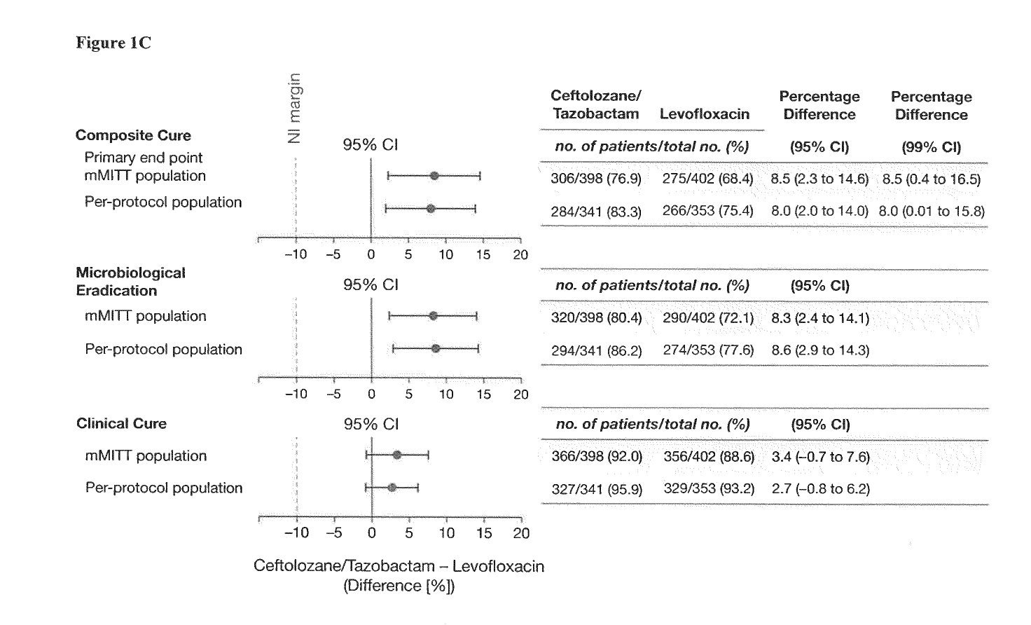

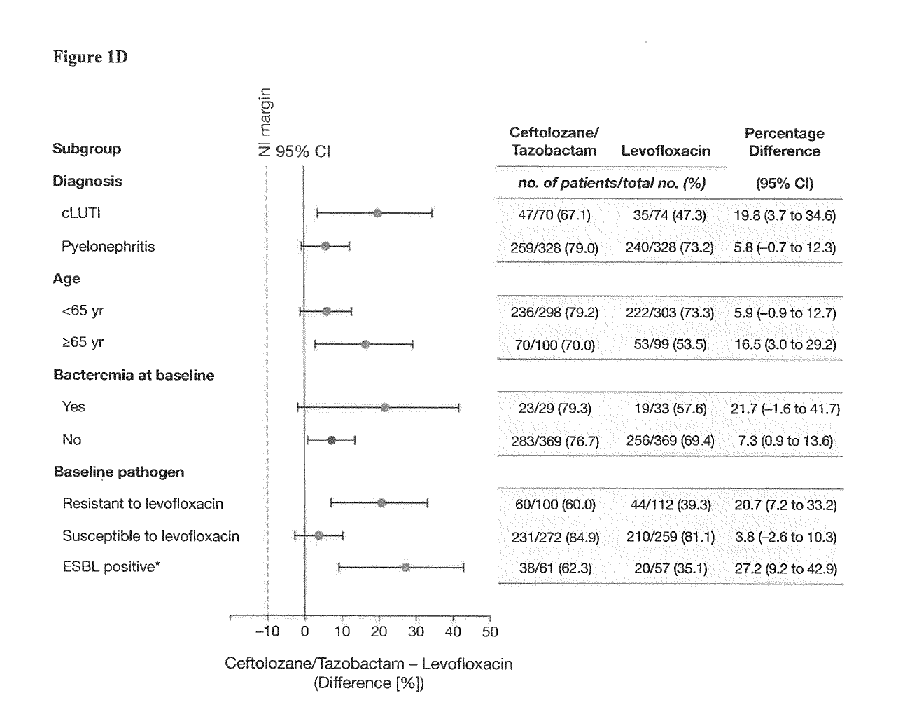

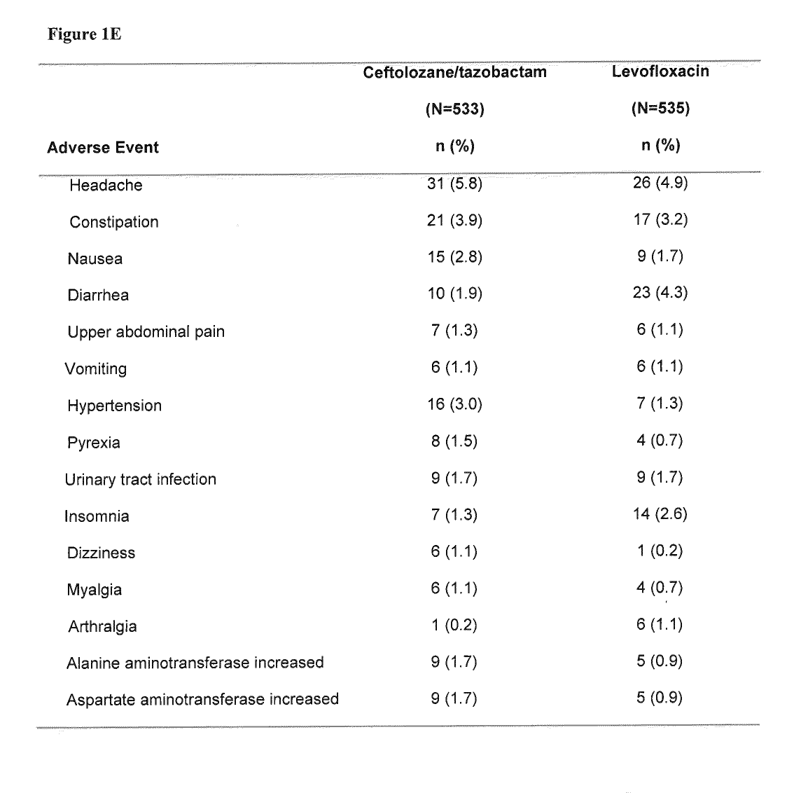

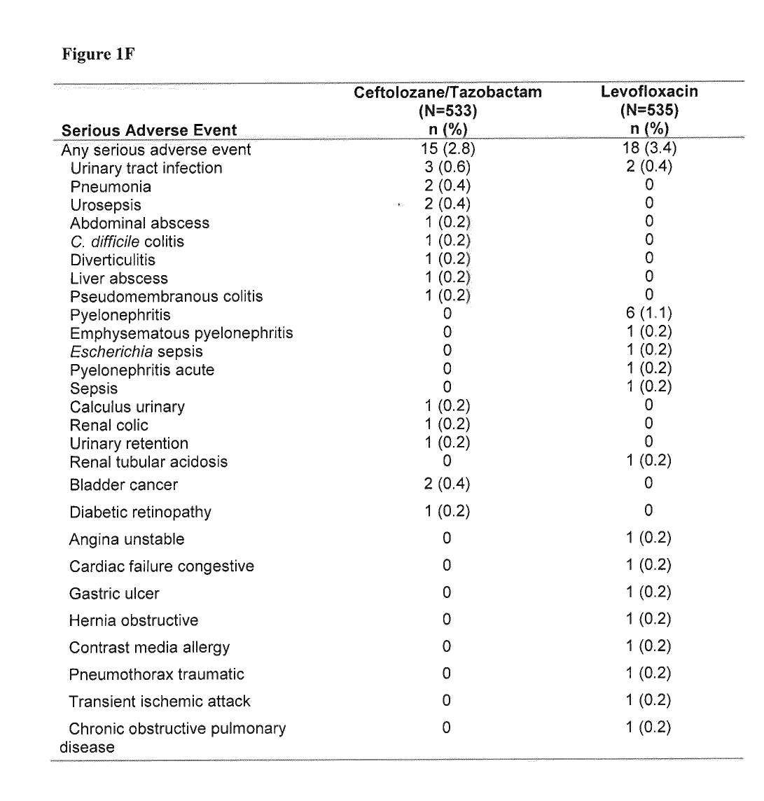

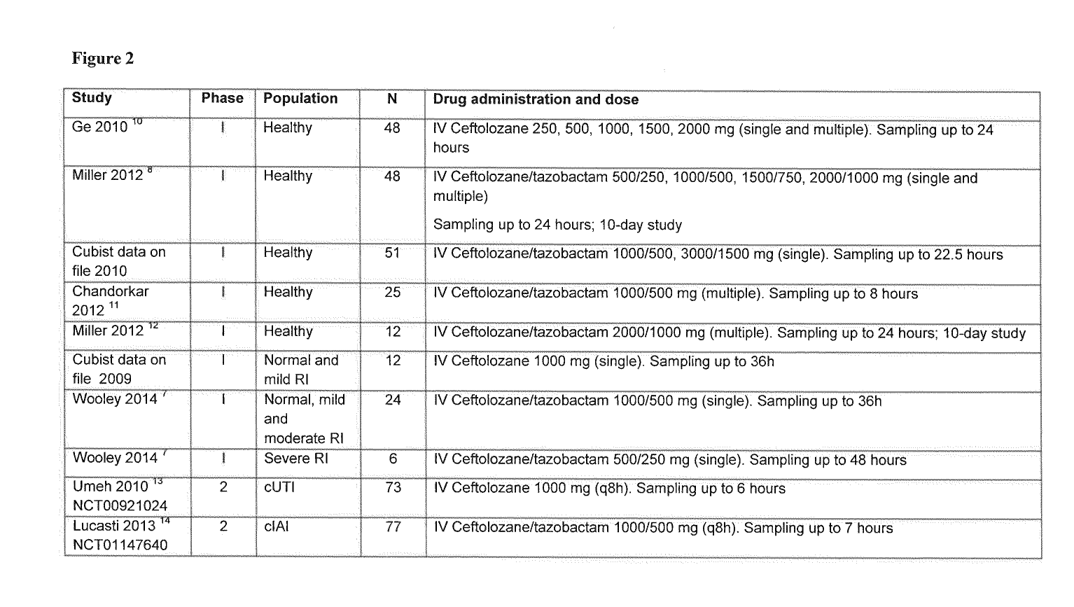

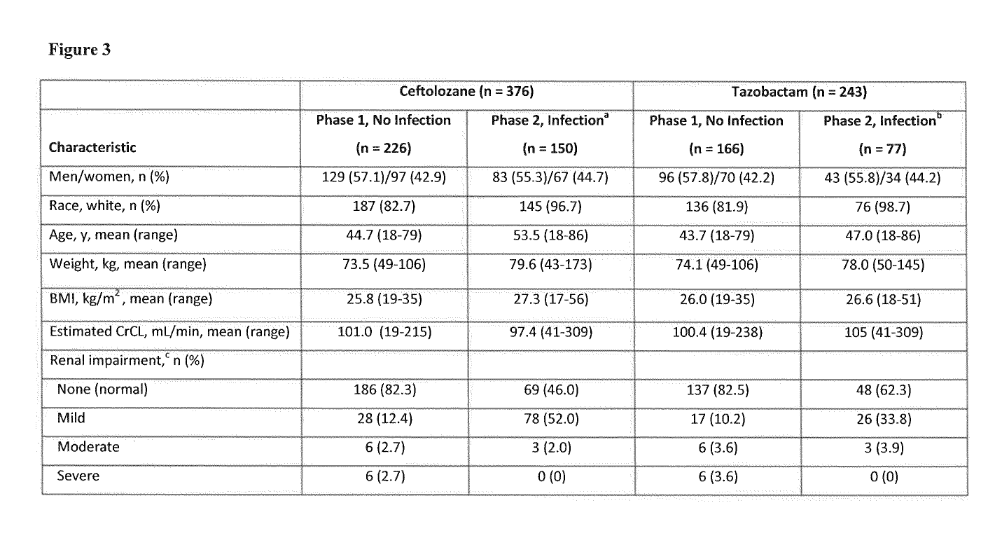

The mMITT population consisted of 800 patients with cUTI, including 656 (82%) with pyelonephritis, 34.3% had mild or moderate renal impairment, and 24.9% were aged ≧65 years. The median age in this population was 50.5 years and 74% were female (FIG. 1A).

Most patients in the mMITT population had a monomicrobial infection (97.0%), most commonly due to E. coli (78.6%). Other baseline uropathogens included Klebsiella pneumoniae (7.3%), Proteus mirabilis (3.0%), and P. aeruginosa (2.9%). In the mMITT population, 26.5% (212/800) of patients had levofloxacin-resistant uropathogens and 14.8% (118/800) had ESBL-producing Enterobacteriaceae organisms isolated from the baseline urine culture.

The results of baseline susceptibility testing to both study drugs are provided in FIG. 1B. In the mMITT population, 96.6% of all gram-negative pathogens isolated at baseline were susceptible to ceftolozane/tazobactam (using a breakpoint of ≦8 mg/1) compared with 70.7% susceptible to levofloxacin using CLSI criteria. 11 Of note, 99.7% of E. coli isolates were susceptible to ceftolozane/tazobactam, regardless of ESBL phenotype (minimum inhibitory concentration required to inhibit the growth of 50%/90% of organisms [MIC50/90] 0.25/0.5 mg/1) compared with 74.1% for levofloxacin (MIC50/90 0.03/>4 mg/1).

CXA-201 was superior to levofloxacin with regard to the composite microbiological and clinical cure rates at the TOC visit in both the mMITT and ME populations (Table 8 and FIG. 1C).

Microbiological eradication rates at the TOC visit by pathogen in the ME population are presented in Table 9A.

In patients with levofloxacin-resistant pathogens at baseline, CXA-201 was superior to levofloxacin with regards to composite cure rates in the mMITT population, 60/100 (60%) in the CXA-201 treatment arm and 44/112 (39.3) in the levofloxacin treatment arm (95% CI: 20.7 [7.23, 33.17]) (Table 9B).

TABLE 8
Composite Microbiological and Clinical Cure Rates in a Phase 3 Study of
Complicated Urinary Tract Infections

| | | | Treatment |
|---|---|---|---|
| Analysis | CXA-201^{a} | Levofloxacin^{b} | Difference |
| Population | n/N (%) | n/N (%) | (99% CI)^{c} |
| mMITT | 306/398 (76.9) | 275/402 (68.4) | 8.5 (0.36, 16.46) |
| ME | 284/341 (83.3) | 266/353 (75.4) | 8.0 (0.01, 15.84) |

^{a}1.5 g IV every 8 hours
^{b}750 mg IV once daily.
^{c}The 99% CI was based on the stratified Newcombe method.

TABLE 9A
Per Pathogen Microbiological Eradication Rates in a Phase 3 Study of
Complicated Urinary Tract Infections (ME Population)

| Organism Group | CXA-201 | Levofloxacin |
|---|---|---|
| Pathogen | n/N (%) | n/N (%) |
| Aerobic Gram-negative | 287/323 (88.9) | 263/340 (77.4) |
| Escherichia coli | 237/262 (90.5) | 226/284 (79.6) |
| Escherichia coli (ESBL-producing) | 27/36 (75) | 18/36 (50) |
| Escherichia coli (CTX-M-14/15 | 20/27 (74.1) | 13/25 (52.0) |
| ESBL producing) | | |
| Klebsiella pneumoniae | 21/25 (84.0) | 14/23 (60.9) |
| Klebsiella pneumoniae (ESBL- | 7/10 (70) | |
| producing) | 2/7 (29) | |
| Klebsiella pneumoniae (CTX-M- | 5/8 (62.5) | 1/4 (25.0) |
| 15 ESBL producing) | | |
| Proteus mirabilis | 10/10 (100) | 8/11 (72.7) |
| Pseudomonas aeruginosa | 6/7 (85.7) | 7/12 (58.3) |

TABLE 9B
Per Pathogen Microbiological Eradication Rates vs Levofloxacin in the
mMITT and ME Populations

| Outcomes in the | | | | |
|---|---|---|---|---|
| Levofloxacin- | | Ceftolozane/ | | |
| resistant | | tazobactam | Levofloxacin | Difference |
| Population at TOC | Population | % (n/N) | % (n/N) | % (95% CI) |
| Composite | mMITT | 60.0 | 39.3 | 20.7 (7.23 |
| Cure Rate | | (60/100) | (44/112) | to 33.17) |
| | ME | 64.0 | 43.4 | 20.6 (6.33 |
| | | (57/89) | (43/99) | to 33.72) |
| Per-pathogen | ME | | | |
| Microbiological | | | | |
| Eradication Rate | | | | |
| Enterobacteriaceae | | 71.4 (55/77) | 45.2 | 26.2 (10.96 |
| | | | (38/84) | to 39.72) |
| Escherichia coli | | 72.9 (43/59) | 44.1 | 28.8 (11.59 |
| | | | (30/68) | to 43.55) |
| Klebsiella | | 81.8 (9/11) | 30.0 (3/10) | 51.8 (9.50 |
| pneumoniae | | | | to 75.05) |
| Pseudomonas | | 100.0 (3/3) | 37.5 (3/8) | 62.5 (-2.09 |
| aeruginosa | | | | to 86.32) |

In the mMITT population, composite cure rates in patients with concurrent bacteremia were 23/29 (79.3%) for CXA-201 and 19/33 (57.6%) for levofloxacin (FIG. 1D).

The incidence of adverse events, including serious adverse events, was low and comparable in both treatment groups (FIGS. 1E and 1F). Adverse events occurred in 34.7% (185/533) and 34.4% (184/535) of patients in the ceftolozane/tazobactam and levofloxacin groups, respectively. The most common adverse events were headache (5.8% and 4.9%) and gastrointestinal symptoms (11.8% and 11.4%) for ceftolozane/tazobactam and levofloxacin, respectively. The majority of adverse events were mild to moderate in severity, and the incidence of treatment-limiting adverse events was <2% in both treatment groups. Epidemiology and susceptibility of organisms isolated from patients with cUTI

### Methods

764 isolates of Gram-negative aerobic organisms were isolated from 800 patients in the microbiological modified intent-to-treat population (enrolled in 20 countries in Eastern Europe, North America, South America and 5 countries outside of these regions). Susceptibility (S) testing was performed with ceftolozane/tazobactam at a fixed 4 μg/mL of tazobactam and 10 antibiotic comparators using CLSI broth microdilution methods. ESBL enzymes were identified by PCR.

### Results

The activity of ceftolozane/tazobactam was similar across geographic regions with the exception of decreased activity against a subset of P. aeruginosa isolates from E. Europe possessing carbapenemases. Potent activity was observed against isolates of Enterobacteriaceae and P. aeruginosa, with limited Enterococcus spp. activity. E. coli was the most common pathogen (78.6%) and ceftolozane/tazobactam was the most active beta-lactam tested against E. coli (99.7% inhibited at ≦8 μg/mL), including CTX-M-14/15+ isolates (MIC90, 1 μg/mL). The majority (60.0%) of P. aeruginosa isolates were inhibited at ceftolozane/tazobactam concentrations ≦8 μg/mL while these isolates exhibited moderate susceptibility to imipenem (IMI, 45.0%), ceftazidime (CAZ; 40.0%), cefepime (FEP, 50.0%), piperacillin/tazobactam (P/T; 40.0%), levofloxacin (LVX; 35.0%), and amikacin (AMI; 55.0%). Against Klebsiella pneumoniae (KPN), ceftolozane/tazobactam activity was improved relative to that of CAZ (87.3% vs 58.2% inhibited at ≦8 μg/mL respectively) including CTX-M-15+ isolates of KPN (73.3% inhibited at ≦8 μg/mL) while the activity of comparators except colistin and IMI was greatly reduced.

### Example 6

### ESBL-Producing Strains of Gram-Negative Pathogens in the Phase 3 Clinical Trials

The clinical response rates of CXA-201 and comparators against E. coli and K. pneumoniae strains producing CTX-M-14/15 ESBLs in the Phase 3 clinical trials are shown in Table 10.

TABLE 10
Clinical Cure Rates by ESBL Status from the Phase 3 Clinical Trials (ME
Population)

| | CXA-201^{a} | All Comparators^{b} |
|---|---|---|
| Pathogen | n/N (%) | n/N (%) |
| Escherichia coli | 452/470 (96.2) | 483/515 (93.8) |
| Escherichia coli (ESBL-producing) | 49/50 (98.0) | 48/56 (87.5) |
| Escherichia coli (CTX-M-14/15 | 35/36 (97.2) | 28/34 (82.4) |
| ESBL-producing) | | |
| Klebsiella pneumoniae | 51/55 (92.7) | 41/48 (85.4) |
| Klebsiella pneumoniae (ESBL- | 17/18 (94.4) | 8/11 (72.7) |
| producing) | | |
| Klebsiella pneumoniae (CTX-M-15 | 13/13 (100) | 2/5 (40.0) |
| ESBL-producing) | | |

^{a}The CXA-201 dose received was 1.5 g IV every 8 hours. In the complicated intra-abdominal infection studies CXA-201 was combined with metronidazole.
^{b}Comparators included meropenem 1 g IV every 8 hours in the Phase 3 complicated intra-abdominal infection trial and levofloxacin 750 mg IV every 24 hours in the Phase 3 complicated urinary tract infection trials

### Example 7

### Population Pharmacokinetics of Ceftolozane/Tazobactam (as CXA-201) in Healthy Volunteers, Subjects with Varying Degrees of Renal Function and Patients with Bacterial Infections

Ceftolozane/tazobactam is a novel antibacterial with potent in vitro activity against Pseudomonas aeruginosa, including drug-resistant strains, and other common Gram-negative pathogens including most extended-spectrum β-lactamase (ESBL)-producing Enterobacteriaceae. Ceftolozane exerts its bactericidal activity by inhibition of essential penicillin-binding proteins (PBPs). Tazobactam is an inhibitor of most common class A β-lactamases and some class C β-lactamases that, by binding to the active site of these enzymes, protects ceftolozane from hydrolysis and broadens coverage to include most ESBL-producing Enterobacteriaceae. In addition, ceftolozane/tazobactam has the most potent anti-pseudomonal activity among currently available cephalosporins and is minimally affected by AmpC overexpression, increases in efflux mechanisms, and porin deficiencies. Ceftolozane/tazobactam is currently in clinical development for the treatment of complicated urinary tract infections (cUTIs), complicated intra-abdominal infections (cIAIs), and nosocomial pneumonia.

The pharmacokinetic (PK) profile of ceftolozane/tazobactam has been studied in several preclinical and clinical studies. In healthy volunteers the PK of ceftolozane/tazobactam is dose-proportional and linear across a wide range of doses (up to 3000 mg/1500 mg as a single dose) with a terminal elimination half-life (t_{1/2β}) of approximately 2.5 hours for ceftolozane and 1 hour for tazobactam. Both ceftolozane and tazobactam are primarily excreted in the urine; ceftolozane almost completely in the urine as unchanged parent drug suggesting minimal metabolism, and tazobactam with 80% as the unchanged parent drug and the remaining as inactive M1 metabolite that is formed via hydrolysis of tazobactam. There is no drug-drug interaction between ceftolozane and tazobactam when co-administered.

PK/pharmacodynamic (PD) models are of particular importance for describing the efficacy and safety of anti-bacterials and for identifying patient covariates that need to be taken into account for determining optimal dose strategies and evaluating exposure-response relationships. The aims of this analysis were: (1) to develop a population PK model for ceftolozane/tazobactam in healthy subjects and in target populations such as patients with renal impairment and complicated bacterial infections; (2) to identify intrinsic and extrinsic determinants of variability (covariates) in the PK of ceftolozane and tazobactam. The analysis was performed using published guidance from the US Food and Drug Administration (FDA) and European Medicines Agency (EMA).

### Materials and Methods

A population-PK analysis was performed on plasma ceftolozane and tazobactam concentration-time data from adult subjects enrolled in 10 studies. Subjects were included from multiple sites and all studies were performed in accordance with the International Conference on Harmonization guidelines on good clinical practice and the Declaration of Helsinki. An investigational review board approved the study protocols at each site.

Serum concentration data were analyzed from 5 studies in healthy volunteers (n=184), 3 studies in subjects with varying degrees of renal impairment (n=42), and 2 phase 2 studies in patients with bacterial infections (cUTIs [n=73] and cIAIs [n=77]). In all studies, ceftolozane was administered as a 1-hour intravenous infusion either alone or in combination with tazobactam. No substitutions were made to account for these missing data points.

### Population Pharmacokinetic Analysis

Base model development. The model was developed in 2 stages: a preliminary PK model was developed based on datasets from 3 studies. In the current analysis, the structural model was refined using the PK data from 10 studies (including patients with cUTI or cIAI) and the covariate analysis repeated. The results based on this revised model are presented here. A nonlinear mixed-effects model was developed with Phoenix® NLME™ software, version 1.2, 2012 (Certara L.P. Pharsight, St. Louis, Mo.) using first-order maximum likelihood estimation, and a 2-compartment structure model was fitted to the plasmaconcentrationtime data. The First Order Conditional Estimation-Extended Least Squares (FOCE-ELS) engine was used for model fitting. The software R (R Foundation for Statistical Computing, Vienna, Austria, 2013) was used to generate tables of post hoc PK parameters and descriptive statistics.

Models had the form:

Cₚᵢⱼ=C(Dᵢ,tⱼ,θᵢ)+εᵢⱼ

θ=(θᵢ₁, . . . ,θᵢₘ)

where Cₚᵢⱼ; is the concentration at j^{th} time for subject i, Dᵢ represents dosing history for subject i, θᵢ is the vector of m model parameters for subject i, and εᵢⱼ is random error associated with a concentration at the j^{th} time (tⱼ) for subject i.

A variance component, which assumed a log-normal distribution of PK parameters, was used to characterize the between subject variability (BSV) and between occasion variability (BOV) in model parameters using the following equation:

θᵢₙ=θ_{TVn}exp(ηᵢₙ)

(θ₁ . . . ηₘ)˜MVN(0,Ω)

Where θ_{TVn} is the population typical value for the n^{th} PK parameter (eg, clearance), and ηₙ is the individual random effect (η is referred to as ETA hereafter) and occasion random effect on the n^{th} parameter for subject i that jointly follow a multivariate normal distribution (MVN) with mean zero and variance Ω.

Residual unexplained variability was modelled using additive±proportional error models, including:

yᵢⱼ=ŷᵢⱼ*(1+ε₁ᵢⱼ)+ε₂ij

where yᵢⱼ (observed) and ŷᵢⱼ (predicted) represent the j^{th} plasma drug concentration for the i^{th} subject, and ε is the random residual variability. Each ε(ε₁ and ε₂) is normally distributed with mean 0 and variance σ.

### Sources of Variability and Covariate Analysis

Sources of variability, that may affect drug exposure, were identified using correlation plots of individual random effects (ETA with mean 0 and estimated variance _{ω}) of parameters such as systemic clearance (CL) and central volume of distribution (Vc) versus covariates. Extrinsic covariates analyzed included dose levels, drug-drug interactions between ceftolozane and tazobactam, and disease status (bacterial infections). Intrinsic covariates analyzed included body weight, age, gender, ethnicity, and baseline calculated CrCL). The CrCL was estimated using the Cockcroft-Gault formula:

CrCL={[(140−Age)×WT]/S_{Cr}}

where CrCL is creatinine clearance (ml/min), age is in years, WT is actual body weight (kg), and S_{Cr} is serum creatinine (mg/dl); for female subjects the value was multiplied by a factor of 0.85. Renal impairment was categorized as normal (CrCL ≧90 mL/min), mild (CrCL ≧50-<90 mL/min), moderate (CrCL ≧30-<50 mL/min), and severe (CrCL ≧15-<30 mL/min).

Scatter plots were used to examine the effect of continuous variables and box plots were used for categorical variables. The resulting graphs were screened using visual inspection, and the most statistically relevant covariates were retained and evaluated in the population PK model using an automatic stepwise forward additive-backward elimination approach to identify individual covariates that had a sufficient threshold effect based on the specified criteria (P<0.01 for forward approach and P<0.001 for backward approach). Covariates were introduced in a multiplicative order using a power model standardized by the median for continuous covariates and a linear model with an exponentiated factor relative to the reference for categorical covariates.

Final population PK model: evaluation and performance. The final population-PK models for ceftolozane and tazobactam were evaluated using standard diagnostics, goodness-of-fit criteria, nonparametric bootstrap resampling, and visual predictive check (VPC). Final model selection was based on goodness-of-fit criteria evaluated using the log-likelihood difference between models, pertinent graphical representations of plasma concentrations (fitted, observed [individual dependent variable], population-predicted [PRED], and individual-predicted [IPRED]) versus time plots with assumption of log-normal distribution of PK parameters for BSV and BOV. Sensitivity of outliers was measured using conditional weighted residuals (CWRES) versus time or time after dose (for FOCE) plots. Shrinkage of individual random effects (ETA) toward the population mean was computed to assess whether the final model provided reliable estimates of individual PK parameters: Shrinkage=1−(SD(ETA)/ω). SD(ETA) is the standard deviation of the post hoc or empirical Bayesian estimates of ETA and ω is the population model estimate of the SD of ETA. Smaller shrinkage ≦0.2 indicates good individual estimates. A VPC was performed to allow for comparisons of simulated and original data. The plasma concentration-time profiles of ceftolozane and tazobactam were simulated using 1000 replicates of the subject, and the median 90% prediction intervals (PI) were computed and compared with observed data. In addition, the robustness of the final population PK model was confirmed using nonparametric bootstrap resampling. The final model was fitted to a 1000 bootstrap dataset to obtain the median value of each PK parameter, along with the fixed-effect and random-effect parameters (interindividual variability and residual error). The nonparametric bootstrap values (median) for each parameter were compared with the original parameter estimates to examine bias and predictive error and were evaluated using 95% confidence intervals (CIs).

### Results

### Data Sets

The population-PK model included evaluable data from 376 adults who received ceftolozane and 243 who also received tazobactam, with 5048 observations for ceftolozane and 4249 observations for tazobactam. Demographic data stratified by presence or absence of infection are summarized in FIG. 3. Approximately, 39.9% (150/376) of subjects included in the PK model had an infection (cUTI or cIAI). Overall, the majority of subjects had normal renal function and 32.2% (121/376) were renally impaired. Mild renal impairment was present in approximately 50% of cUTI patients and 40% of cIAI patients. The age range of subjects was from 18 to 86 years.

### Population Pharmacokinetic Model and Covariate Analysis of Ceftolozane

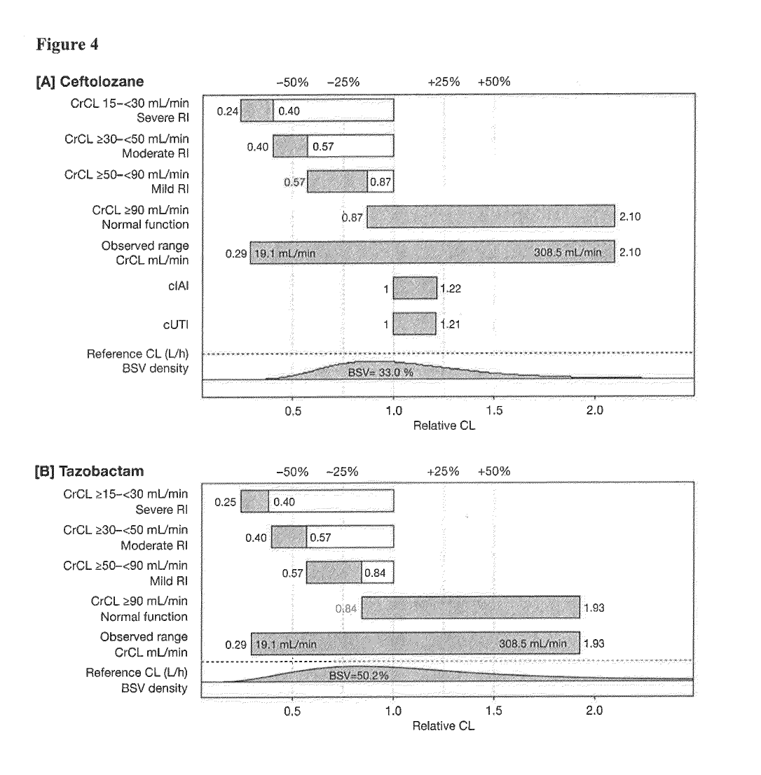

A 2-compartmental structural model with a diagonal variance (omega) of CL, Vc, peripheral volume of distribution (Vp), and peripheral clearance (CL₂) fixed to a value of 0 provided the best data fit. The residual variability was found to be composite (both proportional and additive). Covariate analysis showed that both CL and Vc increased with body weight. A small negative trend between age and CL was also observed but it was not clinically meaningful. Both CL and Vc were significantly different for patients with an infection compared with healthy volunteers, and ceftolozane CL decreased as baseline CrCL decreased. Other covariates such as race, gender, dose level, and drug-drug interaction did not significantly affect CL or Vc of ceftolozane. The stepwise approach to identify significant covariates showed that the greatest improvement in the model included the effect of infection on both CL (<0.001) and Vc (<0.001), body weight on Vc (<0.001), and CrCL on CL (<0.001), with a significant difference between the minimum objective function value for the tested and base models [ΔMOF2] of −329.81; P<0.001). The effects of renal impairment and infection status on ceftolozane CL are presented in a tornado plot (FIG. 4a). The plot shows that between-subject variability (BSV 33.0%) had more impact on relative CL than the effect of infection (cIAI or cUTI). Furthermore, severe renal impairment and moderate renal impairment (based on CrCL categories over a standardized range of 19.1-308.5 mL/min) resulted in lower CL compared with normal and mild renal impairment.

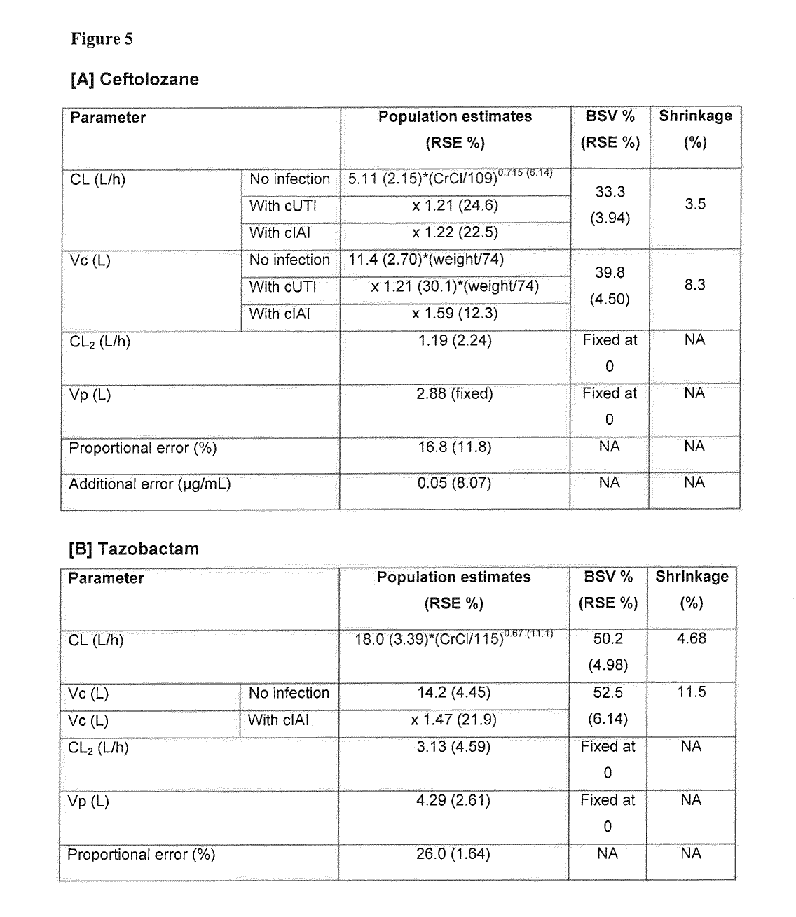

The final model was further refined with infection status divided into cUTI and cIAI. Overall, the refined final model for ceftolozane was a 2-compartment model with linear elimination including the effect of baseline CrCL on CL and body weight on Vc, and the effect of cUTI and cIAI infection on both CL and Vc. The population PK estimates, relative standard error (RSE), and BSV of the model are shown in FIG. 5a. In the final refined model, the Vc changed proportionally (linearly) with body weight in subjects without cIAI. However, in cIAI patients, there was no significant correlation between Vc and body weight given the large observed variability. In addition, CL was similar in patients with cUTI and cIAI (6.18 vs 6.23 L/h at CrCL=109 mL/min), both about 20% higher than that in healthy subjects. Vc was about 30% different between these 2 patient groups (13.8 L at 74 kg body weight for cUTI vs 18.2 L for cIAI). The inter-compartment clearance (CL2) was about 1 L/h while volume of distribution in the peripheral compartment was about 3 L. The parameter estimates of the final model were reliable with all standard error of measurement (SEM %) less than 50%, and the residual variability (ie, the sum of all variability that is not explained by the final model) was low, 16.8% for proportional error and 0.05 μg/mL for additive error. For a fitted ceftolozane concentration of 100 μg/mL, the total residual error would be 16.85 μg/mL.

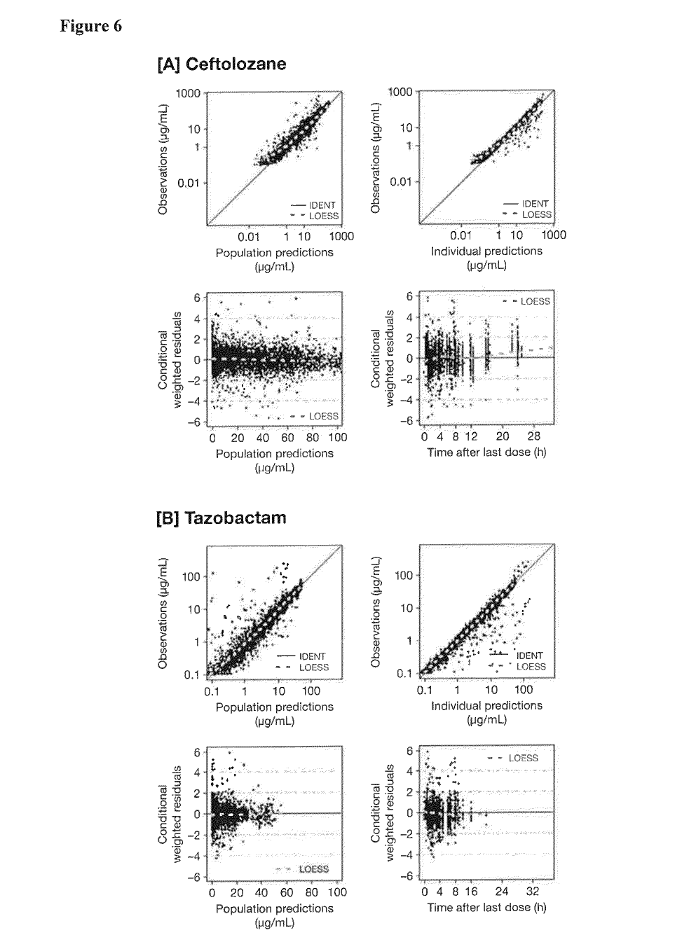

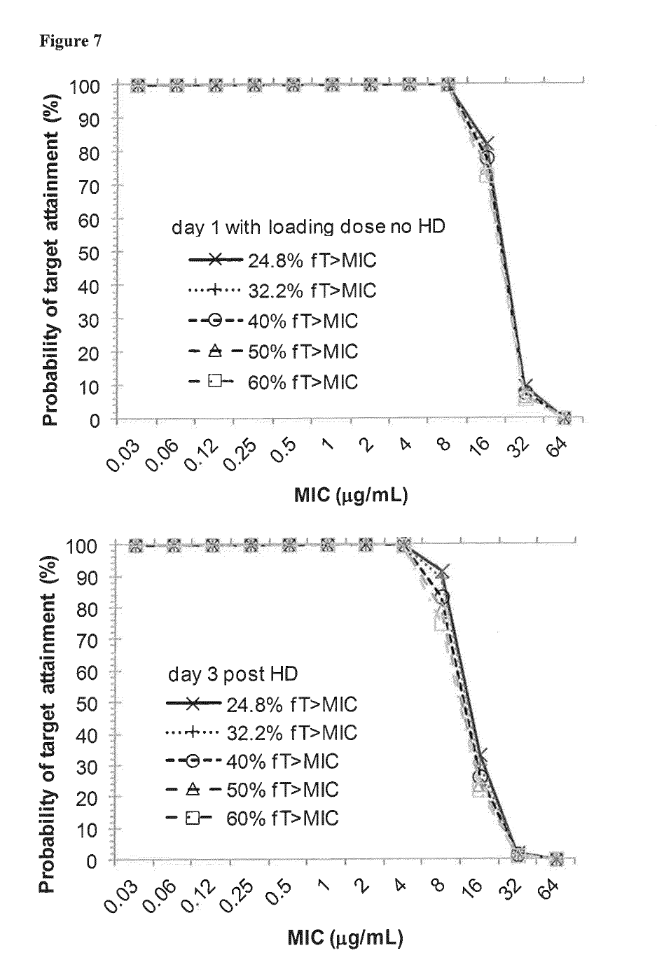

Diagnostic plots showed a good fit of the final model to ceftolozane plasma concentrations (FIG. 6a). Individual observed and PRED plasma concentrations were symmetrically distributed, and CWRES versus PRED were homogenously distributed around 0 with 25 PK samples from 20 subjects displaying CWRES >4, suggesting no bias in predictions relating to low or high ceftolozane concentrations. Outliers (CWRES >4) were not excluded from the analysis, as they did not have a significant effect on PK parameters (difference range: −0.2% to 6.7%) and the changes in BSV of CL and Vc were less than 31%. VPC simulations were within the 90% PI of the predicted median across all doses. Similarly, differences in PK parameters and covariate effects between the final model and bootstrap runs were <5%.

### Population PK Model and Covariate Analysis of Tazobactam

The best-fit model for tazobactam was structurally similar to that for ceftolozane, a 2-compartmental structural model with a diagonal variance (BSV) for CL and Vc and a proportional model for unexplained residual variability. Similar to ceftolozane, in the covariate analysis differences in both CL and Vc were observed between subjects with and without infection, and there was a strong correlation between tazobactam CL and renal impairment category (ie, decrease in CL with decreasing baseline CrCL). The stepwise approach to identify significant covariates showed that the greatest improvement in the model included the effect of cIAI infection on Vc (note there were no tazobactam data from cUTI patients) and of CrCL on CL (ΔMOF2: −92.84; P<0.001). The ΔMOF2 was −103.02 (P=0.001) when the effect of cIAI infection was included in the model and −109.73 (P=0.01) when weight was included on Vc. No trends were noted between other covariates tested and tazobactam PK.

The final model was confirmed to be a 2-compartmental model with linear elimination that included the effect of baseline CrCL on CL showing a power function of 0.67 (ie, [CrCL/115]^{0.7}) and the effect of infection on Vc. In this model, the population estimates (RSE %) derived for tazobactam were 18.0 L/h (3.39) for CL, 14.2 L (4.45) for Vc in subjects without infection, 3.13 L/h (4.59) for CL₂ (inter-compartment clearance), and 4.29 L (2.61) for Vp (FIG. 5b). The parameter estimates of the final model were reliable with all SEM % less than 50%, and a proportional unexplained error of 26.0% (1.64), although the BSV was higher (50.2% for CL and 52.5% for Vc). The tornado plot shows that, similar to ceftolozane, severe and moderate renal impairment resulted in lower CL of tazobactam compared with normal and mild renal impairment (FIG. 4b).

The model was robust showing a good fit to plasma concentrations of tazobactam (FIG. 6b) and CWRES versus PRED were homogenously distributed around 0 with 17 PK samples from 13 subjects displaying CWRES >4 and nonexclusion of outliers (difference range: −2.8% to 7.7%). However, when outliers were excluded the BSV of CL and Vc decreased by 42.5 and 32.5%, respectively. VPC simulations were within 90% PIs of predicted medians and differences in bootstrap resampling analysis were <4% compared with the final model.

### Conclusion

In summary, this analysis conducted by combining PK data across a range of subjects provided a comprehensive, stable, and interpretable model explaining the determinants of variability in the disposition of ceftolozane/tazobactam. The final PK models adequately described the plasma concentrations of ceftolozane and tazobactam and form the basis for evaluation of the probability of target attainment in a diverse population with varying demographics and degrees of renal impairment. For both ceftolozane and tazobactam that are primarily renally eliminated, clearance was influenced by renal function. Other covariates tested, such as age, body weight, gender, ethnicity and presence of infection, had no clinically relevant effects on clearance. The model can be utilized to further support optimal dosing scenarios to maximize efficacy and safety of ceftolozane/tazobactam for treatment of serious bacterial infections in subjects with varying degrees of renal impairment. Monte Carlo simulations derived with the population PK/PD model can also be utilized to further guide dosing recommendations for ceftolozane/tazobactam in various populations, for different pathogens of interest, and for other indications such as nosocomial pneumonia infection.

### Example 8

### Single Dose Pharmacokinetics of Ceftolozane/Tazobactam (as CXA-201) in Subjects with Severe Renal Impairment and End Stage Renal Disease on Hemodialysis

Methods for reducing the concentration of ceftolozane in the blood of a subject can include maintaining the subject on hemodialysis for a period of 4 hours to remove about 66% of the ceftolozane in the blood of the subject. This method is useful, for example, in treating a patient after an overdose of ceftolozane, and is based in part on the discovery that about 66% of ceftolozane was removed from patients during dialysis (e.g., Example 8).

### Study Design and Objectives

This was a Phase 1, multicenter, prospective, open-label study of 750 mg ceftolozane/tazobactam (as CXA-201) administered IV in male and female adult subjects with severe renal impairment (estimated CL_{CR}<30 mL/min) and subjects with end-stage renal disease (ESRD) on hemodialysis (HD). The primary objective of the study was to determine the PK profile of ceftolozane/tazobactam in subjects with severe renal impairment and subjects with ESRD on HD and to determine the effect of HD on the clearance of ceftolozane/tazobactam.

Subjects with severe renal impairment received a single IV 1-hour infusion of 750 mg ceftolozane/tazobactam on Day 1. Subjects with ESRD had a minimum of 3 months of HD prior to enrollment. Subjects in this cohort received an IV dose of 750 mg ceftolozane/tazobactam as a 1 hour infusion immediately after their first HD session on Day 1 (postdialysis, approximately 72 hours prior to the next HD session) and a second dose of ceftolozane/tazobactam approximately 2 hours before their second HD session on Day 4 of the study. Infusion of ceftolozane/tazobactam was completed approximately 1 hour before the start of HD.

Subjects in both cohorts had urine (if not anuric) and plasma samples collected for determination of levels of ceftolozane, tazobactam and the M1 metabolite of tazobactam; subjects in the ESRD on HD cohort also had dialysate fluid samples collected for PK assessment. Plasma, urine, and dialysate levels of ceftolozane, tazobactam, and its metabolite M1 collected over a prespecified interval were determined by LC/MS/MS assay.

### Results

A total of 12 subjects received 750 mg ceftolozane/tazobactam: 6 subjects with severe renal impairment received a single dose and 6 subjects with ESRD received a dose prior to and following HD; all 12 subjects completed the study. A total of 5 males and 7 females ranging in age from 40 to 76 years were enrolled.

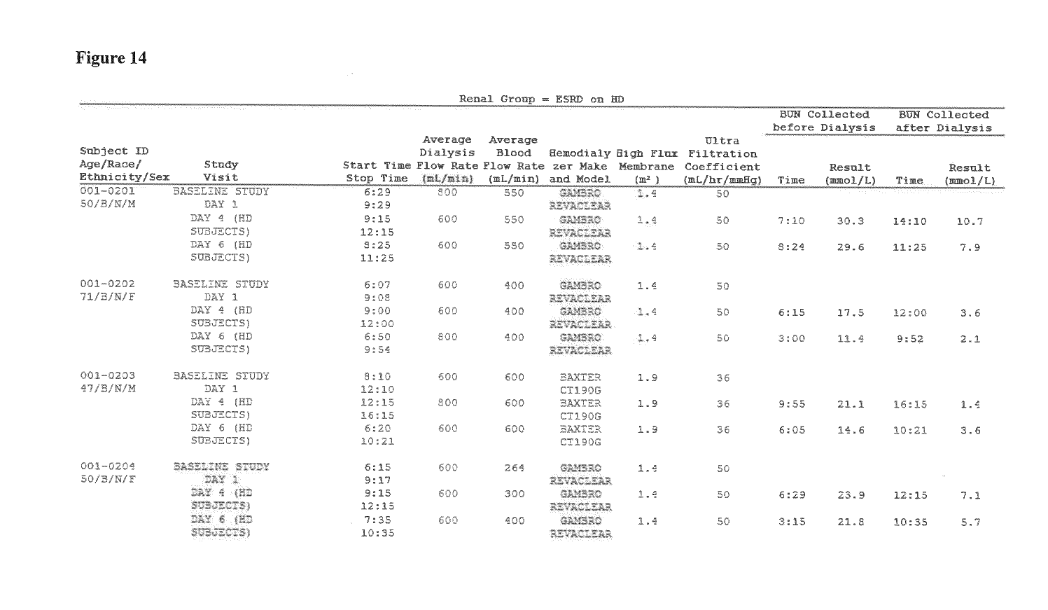

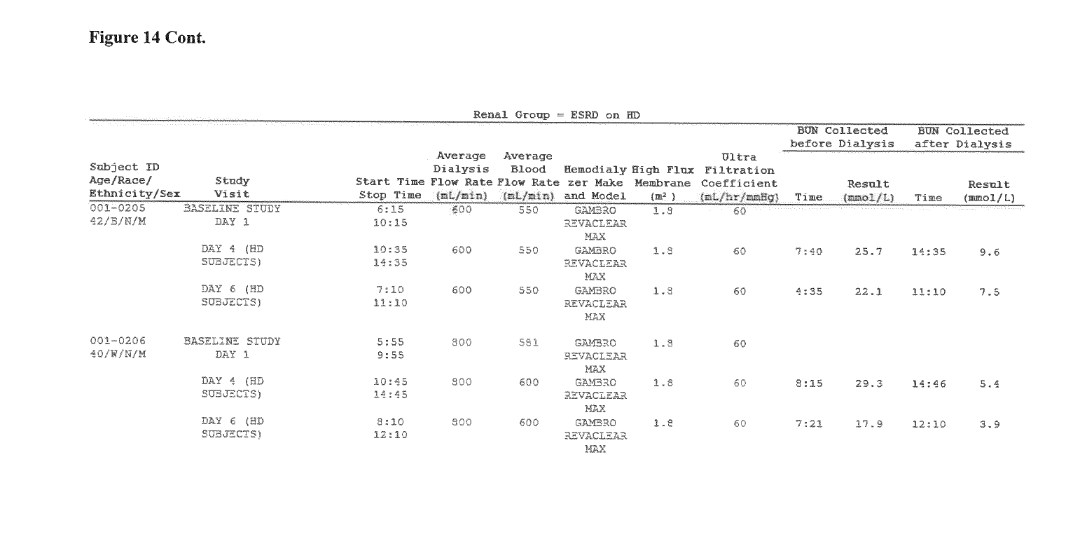

Details regarding the HD procedures for ESRD subjects, including BUN collected before and after dialysis, are provided in FIG. 14. All subjects underwent hemodialysis for 3 to 4 hours using a high-flux membrane (either 1.4, 1.8, or 1.9 m²) on Days 1, 4, and 6 as scheduled. Average blood flow rate was 264 to 600 mL/min and average dialysis flow rate was either 600 or 800 mL/min for all subjects with ESRD.

The PK parameters of ceftolozane, tazobactam, and the M1 metabolite of tazobactam were consistently higher in subjects with severe renal impairment (Table 11) when compared with healthy subjects or subjects with mild or moderate renal impairment from previous studies.

TABLE 11
Plasma Pharmacokinetic Parameters for Ceftolozane, Tazobactam, and
Tazobactam M1 Metabolite in Subjects with Severe Renal Impairment After
a Single IV 1-hour Infusion of 750 mg Ceftolozane/Tazobactam

| | Mean (CV %) | | |
|---|---|---|---|
| Pharmacokinetic | Ceftolozane | Tazobactam | M1 Metabolite |
| Parameter | (N = 6) | (n = 6) | (n = 6) |
| Cₘₐₓ (μg/mL) | 49.9 (28) | 15.2 (22) | 2.2 (20) |
| AUCₗₐₛₜ (μg·h/mL) | 511 (22) | 50.3 (26) | 53.7 (25) |
| AUC_{∞} (μg·h/mL) | 537 (23) | 52.4 (27) | 60.3 (30) |
| t_{1/2} (h) | 11.1 (24) | 2.6 (22) | 12.0 (29) |
| Vₛₛ (L) | 13.8 (25) | 16.5 (27) | ND |
| CL (L/h) | 1.0 (20) | 5.1 (29) | ND |

AUC_{∞} = area under the plasma concentration-time curve from time zero to infinity;
AUCₗₐₛₜ = area under the plasma concentration-time curve from time zero to the last measurable concentration (plasma samples were obtained through 48 hours for subjects with severe renal impairment);
CL = total body clearance from plasma;
Cₘₐₓ = maximum (peak) plasma drug concentration;
CV = coefficient of variation;
ND = not determined;
t_{1/2} = half-life;
Vₛₛ = apparent volume of distribution at steady state after intravenous administration

Due to relative increase in exposure, a 4-fold dose reduction to 750 mg ceftolozane/tazobactam every 8 hours and 375 mg ceftolozane/tazobactam every 8 hours is recommended in subjects with moderate and severe renal impairment, respectively, compared to 1.5 g ceftolozane/tazobactam dose in subjects with normal renal function.

The PK parameters for ceftolozane, tazobactam, and the M1 metabolite of tazobactam in subjects with ESRD not being dialyzed (dosed following HD) are summarized in Table 12. The plasma concentrations of the M1 metabolite increased and did not appear to decline over the 12 to 24 hour sampling interval. Therefore, AUC_{∞} and t_{1/2} for the M1 metabolite could not be calculated.

TABLE 12
Plasma Pharmacokinetics Parameters for Ceftolozane, Tazobactam, and
Tazobactam M1 Metabolite in Subjects with ESRD on HD After the Day 1
Intravenous Infusion (During Non-hemodialysis Phase) of 750 mg
Ceftolozane/Tazobactam

| | Mean (CV %) | | |
|---|---|---|---|
| Pharmacokinetic | Ceftolozane | Tazobactam | M1 Metabolite |
| Parameter | (N = 6) | (n = 6) | (n = 6) |
| Cₘₐₓ (μg/mL) | 44.5 (25) | 21.2 (26) | 10.0 (40) |
| AUCₗₐₛₜ (μg·h/mL) | 910 (35%) | 103 (48) | 367 (42) |
| AUC_{∞} (μg·h/mL) | 1678 (44) | 105 (47) | ND |
| t_{1/2} (h) | 39.8 (33) | 5.23 (42) | ND |
| Vₛₛ (L) | 19.2 (36) | 17.0 (35) | ND |
| CL (L/h) | 0.4 (82) | 3.0 (55) | ND |

AUC_{∞} = area under the plasma concentration-time curve from time zero to infinity;
AUCₗₐₛₜ = area under the plasma concentration-time curve from time zero to the last measurable concentration (plasma samples were obtained through 48 hours for subjects with ESRD on HD following Dose 1);
CL = total body clearance from plasma;
Cₘₐₓ = maximum (peak) plasma drug concentration;
CV = coefficient of variation;
ND = not determined;
t_{1/2} = elimination half-life;
V^{ss} = apparent volume of distribution at steady state after intravenous administration

The PK parameters for ceftolozane, tazobactam, and the M1 metabolite of tazobactam in subjects dosed approximately 2 hours prior to initiation of their 3 to 4 hour HD session are summarized in Table 13. The concentrations of all 3 analytes increased following the start of the infusion but declined rapidly at the start of dialysis. The concentrations continued to decline during HD and rebounded slightly at the end of HD followed by a slow decline over the remainder of the sampling interval.

TABLE 13
Plasma Pharmacokinetics Parameters for Ceftolozane, Tazobactam, and the
M1 Metabolite of Tazobactam in Subjects with ESRD on HD After the
Second Intravenous Infusion of 750 mg Ceftolozane/Tazobactam on Day 4
(During HD)

| | Mean (CV %) | | |
|---|---|---|---|
| Pharmacokinetic | Ceftolozane | Tazobactam | M1 Metabolite |
| Parameter | (N = 6) | (n = 6) | (n = 6) |
| Cₘₐₓ (μg/mL) | 40.9 (35) | 15.6 (37) | 10.8 (46) |
| tₘₐₓ (h)^{(a)} | 1.0 (1.0, 1.0) | 1.0 (1.0, 1.0) | 1.5 (0.5, 24.0) |
| AUCₗₐₛₜ (μg·h/mL) | 304 (35) | 38.4 (37) | 176 (40) |
| t_{1/2} (h) | 43.5 (19) | 5.2 (44) | ** |

AUCₗₐₛₜ = area under the plasma concentration-time curve from time zero to the last measurable concentration (plasma samples were obtained through 44 hours for subjects with ESRD on HD following Dose 2);
Cₘₐₓ = maximum (peak) plasma drug concentration;
CV = coefficient of variation;
ESRD = end-stage renal disease;
HD = hemodialysis;
t_{1/2} = elimination half-life;
tₘₐₓ = time to reach maximum (peak) plasma concentration following drug administration.
^{(a)} Median (minimum, maximum) presented.
** N = 1; one subject showed a slow decline.

A separate analysis was conducted to determine the PK parameters of ceftolozane, tazobactam, and the M1 metabolite of tazobactam in subjects on HD following second dose of CXA-201 on Study Day 4 from the start of the infusion to the end of dialysis. This analysis was conducted in order to determine the PK parameters from the start of infusion to the end of dialysis; results are summarized in Table 14.

TABLE 14
Plasma Pharmacokinetics Parameters for Ceftolozane, Tazobactam, and the
M1 Metabolite of Tazobactam in Subjects with ESRD on HD After the
Second Intravenous Infusion of 750 mg Ceftolozane/Tazobactam on Day 4
(Start of Infusion to End of Dialysis)

| | Mean (CV %) | | |
|---|---|---|---|
| Pharmacokinetic | Ceftolozane | Tazobactam | M1 Metabolite |
| Parameter | (N = 6) | (n = 6) | (n = 6) |
| Cₘₐₓ (μg/mL) | 40.9 (35) | 15.7 (37) | 10.6 (49) |
| tₘₐₓ (h)^{(a)} | 1.0 (1.0, 1.0) | 1.0 (1.0, 1.0) | 1.5 (0.5, 1.5) |
| AUCₗₐₛₜ (μg·h/mL) | 87.5 (30) | 28.8 (34) | 23.8 (44) |
| t_{1/2} (h) | 1.25 (30) | 0.98 (32) | 1.54 (48) |

AUCₗₐₛₜ = area under the plasma concentration-time curve for approximately 6 hours from time of start of infusion to the end of dialysis for subjects with ESRD on HD folowing Dose 2;
Cₘₐₓ = maximum (peak) plasma drug concentration;
CV = coefficient of variation;
ESRD = end-stage renal disease;
HD = hemodialysis;
t_{1/2} = elimination half-life;
tₘₐₓ = time to reach maximum (peak) plasma concentration following drug administration.
^{(a)} Median (minimum, maximum) presented

Concentrations of ceftolozane, tazobactam, and the M1 metabolite declined rapidly following the start of HD. The median exposure of ceftolozane/tazobactam (AUC₄₈ₕ) during and after dialysis is shown in Table 15. Approximately 66% of ceftolozane, 56% of tazobactam and 51% of M1 metabolite of tazobactam was removed by dialysis. As a result, a dosing adjustment that replaces the fraction of ceftolozane/tazobactam, removed due to dialysis is recommended in subjects undergoing intermittent dialysis.

TABLE 15
Exposure (AUC₄₈ₕ) of Ceftolozane, Tazobactam, and the M1 Metabolite of
Tazobactam in Subjects with ESRD During and After Hemodialysis (HD)

| | Median (Range) | | | |
|---|---|---|---|---|
| | (μg·h/mL) | | | |
| | After HD | During HD | Ratio | Percent |
| Analyte | (Day 1) | (Day 3) | (Day 3:1) | Removed |
| Ceftolozane | 903 (372 − 1233) | 298 (179 − 437) | 0.34 (0.26 − 0.48) | 66 (52 − 74) |
| Tazobactam | 107 (45.3 − 169) | 37.1 (19.9 − 57.8) | 0.44 (0.26 − 0.53) | 56 (47 − 74) |
| M1 Tazobactam | 389 (99.8 − 538) | 182 (78 − 255) | 0.49 (0.38 − 0.78) | 51 (22 − 63) |
| metabolite | | | | |

Based on the results of this study, it is recommended that ceftolozane/tazobactam be dosed following HD, and in the event of an overdose, a standard 3 to 4 hour HD session with a high flux membrane could lower plasma concentrations of ceftolozane, tazobactam, and the M1 metabolite substantially. As a result, a dosing adjustment that replaces the fraction of ceftolozane/tazobactam removed due to dialysis is recommended in subjects undergoing intermittent dialysis.

The recommended dose in subjects undergoing dialysis is a single loading dose of 750 mg ceftolozane/tazobactam (500/250 mg) administered every 8 hours by IV infusion followed after 8 hours by a 150 mg every 8 hours maintenance dose of ceftolozane/tazobactam (100/50 mg) for the remainder of the treatment period. On HD days, the dose should be administered at the earliest possible time following completion of dialysis. These doses are predicted to provide total daily exposures of ceftolozane/tazobactam that are comparable to exposures in subjects with normal renal function.

### Pharmacokinetic Conclusions

The PK parameters of ceftolozane, tazobactam, and the M1 metabolite of tazobactam were influenced substantially in subjects with severe renal impairment as well as in subjects with ESRD on HD warranting dose adjustment.

### Example 9

### Ceftolozane/Tazobactam (as CXA-201) Dose Optimization in Patients with End Stage Renal Disease (ESRD) Requiring Hemodialysis (HD) Using Population Pharmacokinetics (pPK) and Monte Carlo Simulations (MCS)

### Methods

CXA-201 plasma concentrations from 6 subjects with ESRD following a single dose without HD and a second dose with HD were used to develop a pPK model (Phoenix NLME, Pharsight). MCS was performed (SAS 9.3, SAS Institute) to predict individual C/T concentrations in 5000 subjects to assess the PTA for different dosing regimens and test a range of free-drug time above MIC (fT>MIC) targets, including 24.8% for bacterio stasis, 32.2% for bactericidal activity (1-log kill) as well as higher thresholds for bactericidal effects up to 60% fT>MIC. MCS was run using ceftolozane MIC determined with 4 mg/L tazobactam.

Details regarding the HD procedures for ESRD subjects, including BUN collected before and after dialysis, are provided in FIG. 14. All subjects underwent hemodialysis for 3 to 4 hours using a high-flux membrane (either 1.4, 1.8, or 1.9 m²) on Days 1, 4, and 6 as scheduled. Average blood flow rate was 264 to 600 mL/min and average dialysis flow rate was either 600 or 800 mL/min for all subjects with ESRD.

### Results

A 2-compartment disposition model plus a covariate effect of HD best described the observed CXA-201 plasma concentrations. The key parameter estimates for the final pPK model were: for ceftolozane, clearance (CL) and volume of distribution (Vc) for the central compartment of 0.34 L/hr and 6 L, respectively, with HD increasing CL and Vc by 60- and 4.7-fold, respectively; for tazobactam, CL and Vc of 3.07 L/hr and 11 L, respectively, with HD increasing CL and Vc by 6.6- and 1.5-fold, respectively. PTA exceeded 90% for an MIC up to 8 μg/mL for ceftolozane across all the tested scenarios. Out of all the tested scenarios, the 500 mg/250 mg C/T single loading dose followed by 100 mg/50 mg every 8 hours maintenance dose via 1-hr infusion achieved a >99% PTA against all targets up to an MIC of 8 μg/mL on day 1 and >97% PTA on all other days without HD. The PTA for bactericidal activity on post HD days was 89%.

### Conclusions

Plasma concentrations following CXA-201 infusion in subjects with ESRD on HD can be best described with a 2-compartment disposition model plus a covariate effect of HD on both CL and Vc. In patients with ESRD on HD, a single loading dose of 500 mg/250 mg C/T infused over 1 hour, followed by 100 mg/50 mg every 8 hours infused over 1 hour, preferably at the earliest possible time following completion of each dialysis, achieved a high PTA and was identified as the optimal dose.

### Example 10 Pharmacodynamic Target Attainment Analyses Supporting the Selection of In Vitro Susceptibility Test Interpretive Criteria for Ceftolozane/Tazobactam (as CXA-201) Against Pseudomonas aeruginosa

Modeling is used to show that the dosages recommend for the different renal types is appropriate to hit the PK/PD targets of % T>MIC

The human phase 1 and 2 PK data was used in a monte carlo model to predict the curves at the dosing regimens and the probability that the drug levels would be high enough at the corresponding MIC values.

### Monte Carlo Simulation

Using SAS 9.2 [SAS 9.2 for Windows [computer program]. Cary, N.C.: SAS Institute Inc. 2010] Version Monte Carlo simulation was conducted to generate 5,000 patients, with 1,000 in each of five renal function categories. These categories (and corresponding creatinine clearance (CLcr) ranges) were as follows:
   High normal renal function (150<to £ 200 mL/min);
   Normal renal function (90<to ≦150 mL/min);
   Mild renal impairment (50<to £ 90 mL/min);
   Moderate renal impairment (29≦to ≦50 mL/min); and
   Severe renal impairment (15≦to <29 mL/min).
Using the fixed and random effects parameter estimates and variance-covariance matrix from a previously-developed population PK model for TOL, plasma concentration-time profiles for TOL were generated for simulated patients in each renal function category following selected CXA-201 dosing regimens.
   CXA-201 dosing regimens administered over 1 hr every 8 hrs (q8 h) by renal function category included 1000 mg and 2000 mg TOL adjusted for renal function categories as follows:
   1000 mg TOL regimens:
   1000/500 mg CXA-201 in patients with high normal and normal renal function and patients with mild renal impairment;
   500/250 mg CXA-201 in patients with moderate renal impairment; and
   250/125 mg CXA-201 in patients with severe renal impairment.
   2000 mg TOL regimens:
   2000/1000 mg CXA-201 in patients with high normal and normal renal function and patients with mild renal impairment;
   1000/500 mg CXA-201 in patients with moderate renal impairment; and
   500/250 mg CXA-201 in patients with severe renal impairment.
As the activity of TOL is not enhanced significantly by TAZ due to lack of inhibition of AmpC beta-lactamase [SAS 9.2 for Windows [computer program]. Cary, N.C.: SAS Institute Inc. 2010], only TOL exposures were considered in these analyses.

### PK-PK Target Attainment Analyses

Using non-clinical PK-PD targets, PK-PD TA by MIC was assessed for simulated patients in each renal function category in the context of MIC distributions for CXA-201 against P. aeruginosa based on surveillance data from the United States (US) and the European Union (EU).
Non-clinical PK-PD targets were based on the results from a neutropenic murine-thigh infection model in which P. aeruginosa was evaluated [JMI Laboratories. Surveillance of ceftolozane/tazobactam antimicrobial activity when tested against Gram-negative organisms and streptococci isolated in the USA (2012). Final Report. February 2013; Craig W A, Andes D R. Antimicrob Agents Chemother. 2013; 57:1577-82]
The percentage of the dosing interval that concentrations are above the MIC (% T>MIC) was the PK-PD driver most associated with efficacy for TOL.
For the PK-PD analyses carried out, free-drug (f) % T>MIC targets of 24.8 and 32.2, which were associated with net bacterial stasis and a 1-log₁₀ colony forming units (CFU) reduction from baseline, respectively, and f % T>MIC targets of 40, 50, and 60 were assessed.
The percentage of simulated patients that attained these targets during the dosing interval at steady-state for MIC values ranging from 0.03 to ≧32.2 mg/L was determined for each TOL dosing regimen evaluated within each renal function category.

For patients severe renal impairment administered CXA-201 250/125 mg q8 h a PK-PD MIC cut-off value of 8 mg/L was identified (which is similar to the cutoff predicted for the 1.5 g dose for normal renal function).

The PK-PD MIC cutoff values for CXA-201 1000 and 2000 mg q8 h dosing regimens against P. aeruginosa by renal function category is shown in Table 16.

TABLE 16
PK-PD MIC Cutoff Values for CXA-201 Dosing Regimens Against
P. aeruginosa by Renal Function Category

| | | | % simulated patients |
|---|---|---|---|
| Renal function | TOL/TAZ dosing | MIC | achieving free-drug 5T>MIC |
| category^{A} | regimen (mg)^{B} | (mg/L)^{C} | targets 24.8/≧32.2^{D} |
| High normal | 1000/500 | 4 | 99.5/96.1 |
| | 2000/1000 | 8 | 99.5/96.1 |
| Normal | 1000/500 | 8 | 99.1/94.7 |
| | 2000/1000 | 16 | 99.1/94.7 |
| Mild | 1000/500 | 8 | 100/99.8 |
| | 2000/1000 | 16 | 100/99.8 |
| Moderate | 500/250 | 8 | 99.9/99.5 |
| | 1000/500 | 16 | 99.9/99.5 |
| Severe | 250/125 | 8 | 98.4/96.1 |
| | 500/250 | 16 | 98.4/96.1 |

^{A}Renal function categories were defined as follows: High normal function = CLcr (mL/min) <150-≦200; Normal renal function = CLcr (mL/min) <90-≦150; Mild renal impairment = CLcr (mL/min) <50-≦90; Moderate renal impairment = CLcr (mL/min) <29-≦50; Severe renal impairment = CLcr (mL/min) <15-≦29.
^{B}TOL/TAZ administration via 1 h intravenous infusion Q8h.
^{C}Represents the highest MIC associated with ≧90% PK-PDTA.

### Example 11

### Impact of Renal Function on the Pharmacokinetics and Safety of Ceftolozane/Tazobactam

The pharmacokinetics (PK) of ceftolozane/tazobactam in patients with normal renal function are linear across a wide range of doses (up to 3,000 mg/1,500 mg as a single dose). Terminal elimination half-lives (t_{1/2}) are approximately 2.5 h for ceftolozane and 1 h for tazobactam. Both compounds exhibit low protein binding (approximately 20% for ceftolozane and 30% for tazobactam) and are primarily excreted in the urine; ceftolozane as unchanged parent drug suggesting minimal metabolism, and tazobactam with 80% as the unchanged parent drug and the remaining as inactive M1 metabolite. In the present studies, the PK and safety of ceftolozane/tazobactam were investigated in subjects with varying degrees of renal function, including subjects with end-stage renal disease (ESRD) on hemodialysis (HD).

### Materials and Methods

### Study Populations

Male and female subjects, aged 18 to 79 years, with varying degrees of renal function were enrolled in two, prospective, open-label, phase I studies of intravenous ceftolozane/tazobactam. A total of 36 subjects were enrolled into cohorts based on degree of renal function: normal (n=11), mild impairment (n=6), moderate impairment (n=7), severe impairment (n=6), and ESRD on HD (n=6). To ensure subjects with ESRD were receiving effective HD, a target adequacy of HD calculated from the pre- and post-blood urea nitrogen ratios (Kt/V) of at least 1.2 for a minimum of 3 months prior to enrollment was required. Renal impairment groups were classified according to the 2010 U.S. Food and Drug Administration draft guidance using creatinine clearance (CrCl) estimated by the Cockcroft-Gault formula (normal impairment, >90 ml/min; mild impairment, 60 to 89 ml/min; moderate impairment, 30 to 59 ml/min; and severe impairment, 15 to 29 ml/min, ESRD <15 ml/min).

### Dosing/Design

All cohorts received ceftolozane/tazobactam as an intravenous infusion over 1 h. The normal, mild, and moderate renal impairment cohorts received a single dose of ceftolozane/tazobactam 1,000 mg/500 mg; the severe renal impairment cohort received a single dose of ceftolozane/tazobactam 500 mg/250 mg; the ESRD cohort received a dose of ceftolozane/tazobactam 500 mg/250 mg initiated at the end of HD on day 1 and another dose initiated 2 h before HD on day 4. Subjects with ESRD underwent HD for 3 to 4 h using a high-flux membrane as scheduled; average dialysis flow rate was 600 to 800 ml/min. Revaclear hemodialyzers (Gambro, Stockholm, Sweden) were used in 5 subjects (ultrafiltration coefficient 50 to 60 ml/b/mmHg, high flux membrane of 1.4 to 1.8 m²) and a CT 190G hemodialyzer (Baxter Healthcare, McGaw Park, Ill.) was used in 1 subject (ultrafiltration coefficient 36 ml/b/mmHg, high flux membrane of 1.9 m²). The average blood flow rate was 400 to 600 ml/min with the exception of one subject with rates between 264 and 400 ml/min.

### Pharmacokinetic Evaluations

Plasma concentrations of ceftolozane and tazobactam were measured prior to, during, and following administration of ceftolozane/tazobactam. Blood samples were collected 30 min prior to administration, at the end of administration and at 5, 15, and 30 min and 1, 2, 3, 5, 7, 9, 11, 15, 25, and 35 h after completion of ceftolozane/tazobactam administration in the normal, mild, and moderate renal impairment cohorts. Severe renal impairment and ESRD cohorts off HD had samples taken 30 min prior to administration and at 0.5, 1, 1.5, 2, 3, 6, 9, 12, 24, 36, and 48 h after the start of administration. On the day of and following HD, the subjects with ESRD had samples taken 30 min prior to administration and at 0.5, 1, 1.5, 2, 3, 4, 5, 6, 9, 12, 24, 36, and 44 h after the start of the administration. The entire dialysate was collected at each of the following intervals: 0 to 1, 1 to 2, 2 to 3, and 3 h to the end of dialysis. Urine for PK analysis was obtained in the normal, mild, and moderate cohorts at 0 to 2, 2 to 4, 4 to 8, 8 to 12, 12 to 24, and 24 to 36 h after the start of ceftolozane/tazobactam administration. In the ESRD and severe renal impairment cohorts, urine was collected during the confinement period pre-dose, 0 to 24 h, and 24 to 48 h after the start of the administration, unless the subject was anuric. A validated LC/MS/MS method was utilized to analyze all plasma, urine, and dialysate samples for ceftolozane and tazobactam (MicroConstants Inc, San Diego, Calif.) (4). The lower limit of quantification (LLOQ) in plasma was 0.25 μg/ml for ceftolozane and 0.1 μg/ml for tazobactam. The assay was linear between 0.25 and 150 μg/ml for ceftolozane and between 0.1 and 50 μg/ml for tazobactam. The precision of the assay for ceftolozane and tazobactam ranged between 3.13 and 7.97% while the accuracy was ±1 and ±6.25%, respectively. The LLOQ in dialysate for both ceftolozane and tazobactam was 1 ng/ml and the assay was linear between 1 and 500 ng/ml. The precision of the assay in dialysate samples for ceftolozane and tazobactam ranged between 1.28 and 9.18% while the accuracy for ceftolozane and tazobactam was ±8.3 and ±9.67%, respectively. The LLOQ for ceftolozane and tazobactam in urine was 5 and 10 μg/ml, respectively, and the assay was linear between 5 and 5,000 μg/ml for ceftolozane and between 10 and 10,000 μg/ml for tazobactam. The precision of the assay for ceftolozane and tazobactam ranged between 3.71 and 9.06% while the accuracy was ±9.20 and 7.33%, respectively.

Pre-dose values below the LLOQ values were set to zero and all missing values below the LLOQ obtained after the first quantifiable concentration were designated as missing and not included in the analysis. The maximum plasma concentration (Cₘₐₓ) and plasma concentration when the last quantifiable concentration was observed relative to the end of infusion (Cₗₐₛₜ) were taken directly from concentration-time data. Terminal elimination t_{1/2} was calculated as 0.693/λ_{z} where λ_{z} is the terminal elimination rate constant, estimated by regression of the terminal log-linear phase of the plasma concentration versus time curve. Area under the plasma concentration time curve (AUC) from time zero to the last measurable concentration (AUC₀₋ₜ) was calculated using the linear trapezoidal rule. The AUC extrapolated to infinity (AUC_{0-∞}) was estimated using the formula AUC₀₋ₗₐₛₜ+(Cₗₐₛₜ/λ_{z}) using the linear trapezoidal rule. Total body clearance from plasma (CL) was calculated as dose/AUC_{0-∞}. Volume of distribution at steady-state (Vₛₛ) was calculated as mean residence time*CL. Renal clearance (CLᵣ) in subjects that provided urine samples was calculated from the equation CLᵣ=Aₑ/AUC_{0-∞} where Aₑ is the cumulative amount of drug recovered in the urine during the sampling period. Dialysis clearance was calculated as the amount of ceftolozane or tazobactam recovered in dialysate divided by AUC from the time of the second dose to the end of HD (AUC₍ₜ₀₋ₜ₁₎). The rate of decrease in plasma concentration (RDHD) was calculated from the difference between the concentration at the end of dialysis (C₂) and the concentration at the beginning of HD (CO. The percent reduction was calculated using the equation RDHD=100*(C₁−C₂)/C₁. Extraction ratio was calculated as 100*(C_{A}−C_{V})/C_{A}where C_{A} and C_{V} are pre- and post-dialyzer paired drug concentrations at the arterial and venous sites. Total effective removal was calculated with individual AUC_{0-∞} values as (AUC_{off-HD}−AUC_{on-HD}) divided by AUC_{off-HD} (6). Dialysis clearance (CL_{D}) was calculated as amount of drug in dialysate divided by AUC₍ₜ₀₋ₜ₁₎.

The PK parameters were calculated by non-compartmental analysis using Phoenix WinNonlin version 6.1 (Pharsight Corporation, Mountain View, Calif.).

### Safety Monitoring

Safety was assessed by monitoring for adverse events (AEs) from the first dose of drug through the last study evaluation, and by review of vital signs, physical examinations, 12-lead electrocardiograms, and clinical laboratory evaluations.

### Results

### Demographics and Disposition

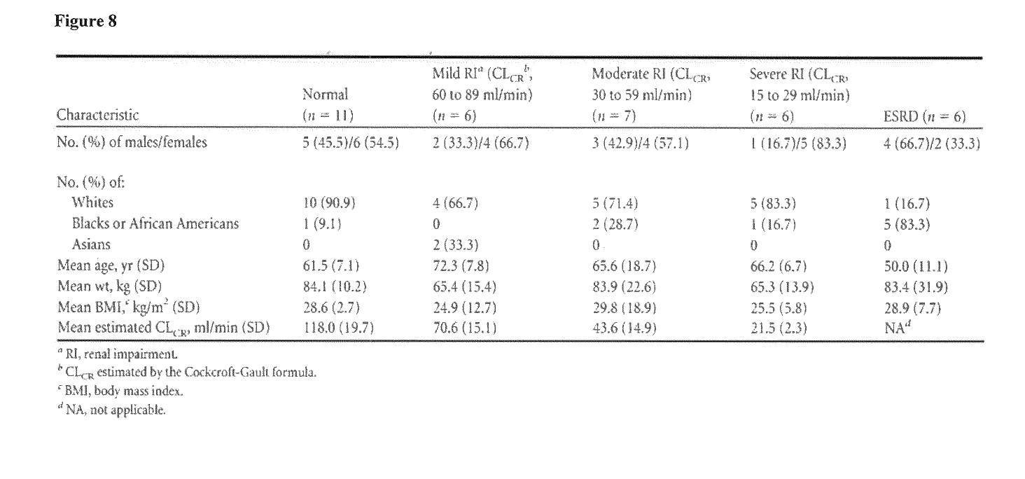

A total of 36 subjects received ceftolozane/tazobactam. No subjects withdrew consent or discontinued due to an AE, and all subjects were included in the PK and safety analyses. The demographic characteristics of the subjects are presented in FIG. 8. The majority were white, except in the ESRD cohort in whom five of the six subjects were black or African American. Subjects ranged in age from 40 to 79 years with a median age of 62 years.

### Pharmacokinetic Summary

### Normal Renal Function and Mild, Moderate, and Severe Renal Impairment

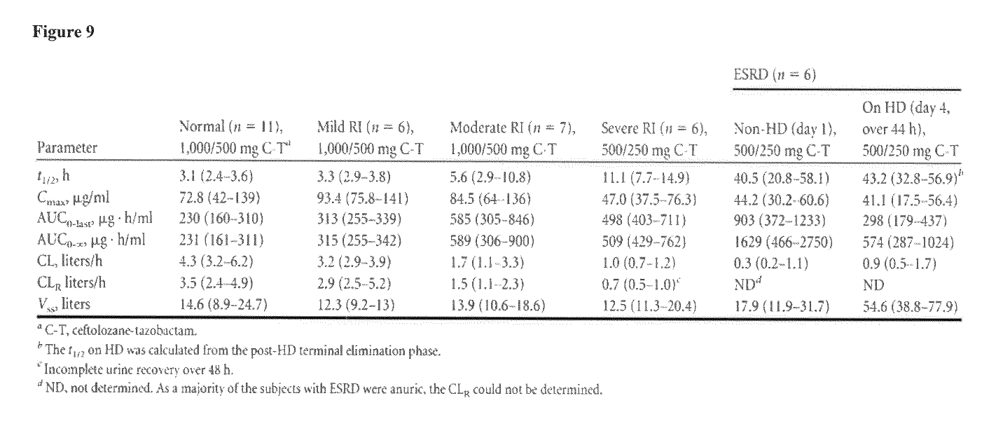

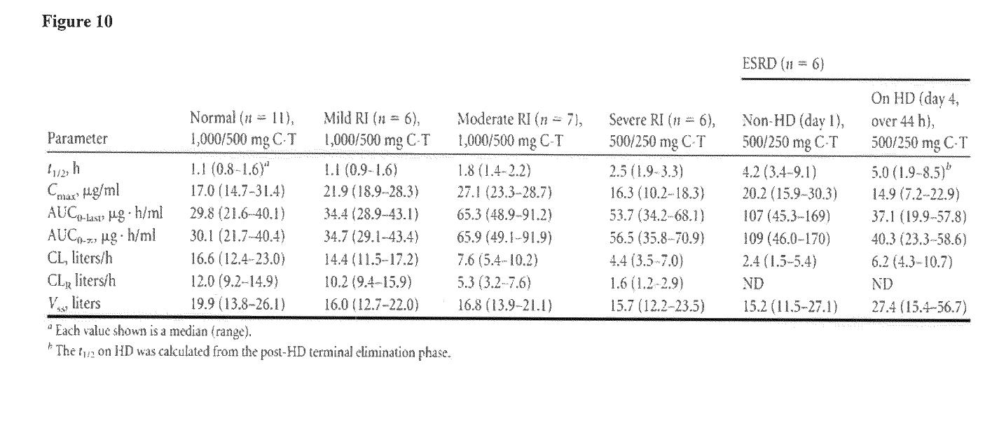

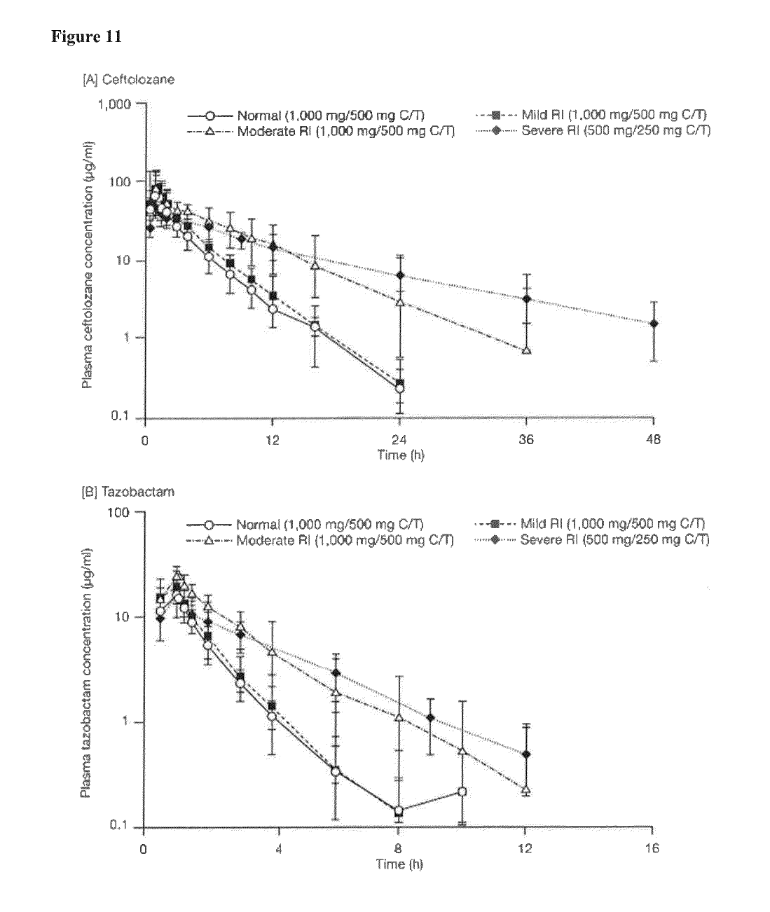

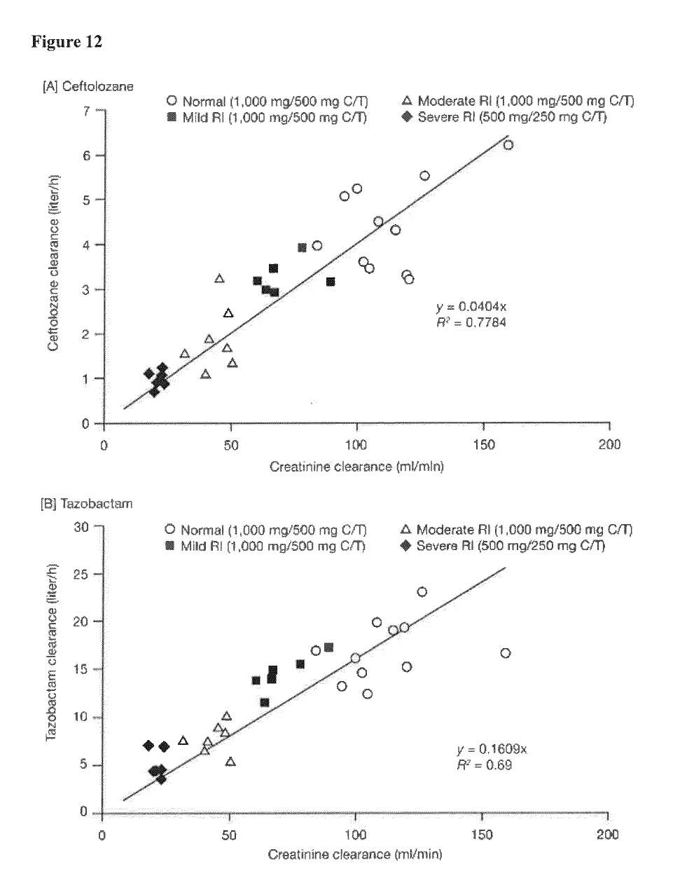

Compared with subjects with normal renal function, the concentration-time profiles of ceftolozane/tazobactam were increasingly altered in subjects with increasingly impaired renal function (FIGS. 11A and 11B). Pharmacokinetic parameters are summarized in FIGS. 9 and 10 for ceftolozane and tazobactam, respectively. Ceftolozane and tazobactam plasma clearance by CrCl are provided in FIGS. 12A and 12B, respectively. Exposure (AUC_{0-∞} and Cₘₐₓ) was similar in subjects with normal renal function and mild renal impairment following a single ceftolozane/tazobactam 1,000 mg/500 mg dose as was t_{1/2} In subjects with moderate renal impairment, decreases in clearance led to increased ceftolozane and tazobactam exposure compared with subjects with normal renal function with median AUC_{0-∞} and Cₘₐₓ, increased for ceftolozane (2.5- and 1.2-fold, respectively) and tazobactam (2.2- and 1.6-fold, respectively). In subjects with severe renal impairment, the median AUC_{0-∞} and Cₘₐₓ, increased 4.4- and 1.3-fold for ceftolozane and 3.8- and 1.9-fold for tazobactam, respectively, compared with the dose-normalized exposure in the normal renal function group.

### End-Stage Renal Disease on Hemodialysis

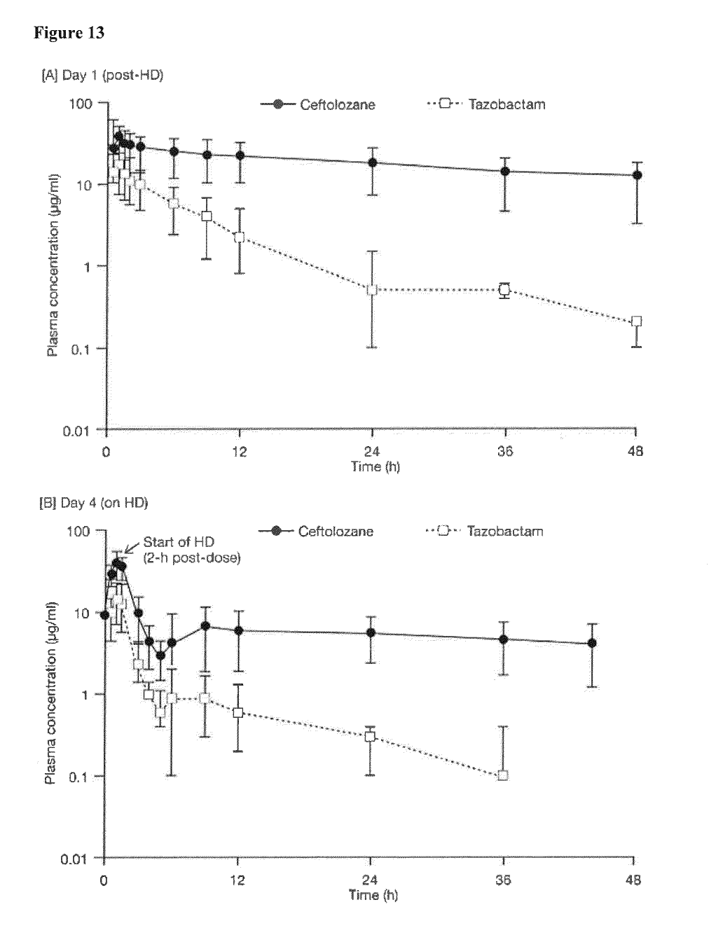

Median concentration-time profiles for ceftolozane and tazobactam in subjects with ESRD post-HD and on HD are shown in FIGS. 13A and 13B, respectively. The PK parameters of ceftolozane and tazobactam differed substantially in subjects with ESRD compared with the other renal impairment groups. Pharmacokinetic parameters are summarized in FIGS. 9 and 10 for ceftolozane and tazobactam, respectively. The median elimination t_{1/2} of ceftolozane and tazobactam in subjects with ESRD during non-HD was prolonged and the median Cₘₐₓ in plasma was 1.2- and 2.4-fold higher compared with subjects with normal renal function when dose normalized. The t_{1/2} during the HD period for ceftolozane and tazobactam were 1.13 and 0.91 h, respectively. The extraction ratios at 1 h and 2 h after the start of HD and at end of HD for ceftolozane and tazobactam were 42, 48, and 47% and 48, 54, and 55%, respectively. The average extraction ratio during HD was 46% (±16) for ceftolozane and 53% (±22) for tazobactam. Ceftolozane and tazobactam concentrations declined rapidly following the start of HD with approximately 66 and 56% reductions in overall exposure to ceftolozane and tazobactam, respectively, based on the AUC_{0-∞} on and off HD. The median RDHD for ceftolozane and tazobactam was 92 and 95%, respectively, indicating significant removal by HD; however, in the period following HD, plasma concentrations rebounded and peaked at approximately 17 and 6% of the original Cₘₐₓ of ceftolozane and tazobactam, respectively. The median CL_{D} for ceftolozane and tazobactam was 5.75 and 4.39 liter/h, respectively.

### Safety

Overall, seven of the 36 subjects experienced a total of 12 AEs. The most common AE reported was headache in three subjects. All events reported were mild in severity with the exception of one event of moderate headache in a subject with normal renal function. Two subjects with normal renal function and one subject with mild renal impairment reported the AE of headache. Diarrhea, infusion-site hemorrhage, and injection-site hemorrhage were reported in one subject each in the mild impairment group. Flatulence, glossodynia, myalgia, and vulvovaginal pain were reported in one subject each in the ESRD on HD group. No AEs were reported in the moderate or severe renal impairment groups. One serious AE of thrombosis of an arteriovenous fistula was reported in a subject with ESRD on HD 7 days after the last dose of the study drug. No subjects withdrew due to AEs. Review of clinical laboratory values, physical examination, and vital signs showed no meaningful changes from baseline.

In summary, the exposure to ceftolozane/tazobactam in subjects with mild renal impairment was increased relative to that in normal controls, but the increase was small and not clinically meaningful, suggesting that no dose adjustment is necessary in this population. However, data from these phase I studies suggest that a decrease in dose or frequency of administration, or both, is necessary in those with moderate or severe renal impairment, or with ESRD.

What is claimed is:

1. A method of treating a complicated intra-abdominal infection or a complicated urinary tract infection in a human patient having a creatinine clearance of less than 15 mL/minute, the method comprising intravenously administering to the patient 500 mg of ceftolozane active and 250 mg tazobactam active, followed by administering one or more additional doses of 100 mg ceftolozane active and 50 mg of tazobactam active to the patient every 8 hours for the duration of a treatment period.

2. The method of claim 1, where the 500 mg of ceftolozane active is co-administered with the 250 mg tazobactam in a single pharmaceutical composition.

3. The method of claim 1, where the 100 mg of ceftolozane active is co-administered with the 50 mg tazobactam in a single pharmaceutical composition.

4. The method of claim 1, wherein the infection is a complicated intra-abdominal infection.

5. The method of claim 4, wherein the method further comprises administering to the patient a therapeutically effective amount of metronidazole.

6. The method of claim 1, wherein the complicated intra-abdominal infection is caused by a microorganism selected from the group consisting of: Citrobacter freundii, Enterobacter cloacae, Escherichia coli, Escherichia coli CTX-M-14 extended spectrum beta-lactamase producing strains, Escherichia coli CTX-M-15 extended spectrum beta-lactamase producing strains, Klebsiella oxytoca, Klebsiella pneumonia, Klebsiella pneumoniae CTX-M-15 extended spectrum beta-lactamase producing strains, Proteus mirabilis, Pseudomonas aeruginosa, Bacteroides fragilis, Bacteroides ovatus, Bacteroides thetaiotaomicron, Bacteroides vulgatus, Streptococcus anginosus, Streptococcus constellatus, and Streptococcus salivarius.

7. The method of claim 1, wherein the complicated urinary tract infection is caused by one of the following Gram-negative microorganisms: Escherichia coli, Escherichia coli levofloxacin resistant strains, Escherichia coli CTX-M-14 extended spectrum beta-lactamase producing strains, Escherichia coli CTX-M-15 extended spectrum beta-lactamase producing strains, Klebsiella pneumoniae, Klebsiella pneumonia levofloxacin resistant strains, Klebsiella pneumonia CTX-M-15 extended spectrum beta-lactamase producing strains, Proteus mirabilis or Pseudomonas aeruginosa.

8. The method of claim 1, wherein the ceftolozane active is provided as ceftolozane sulfate and the tazobactam active is provided as tazobactam sodium.

9. The method of claim 2, wherein the pharmaceutical composition is obtained by reconstituting a mixture comprising a total of 1000 mg ceftolozane active and 500 mg tazobactam active with sterile water for injection or 0.9% Sodium Chloride for injection, USP to form a reconstituted solution and withdrawing a portion of the reconstituted solution and adding it to an infusion bag containing 0.9% Sodium Chloride for Injection, USP or 5% Dextrose Injection, USP.

10. The method of claim 9, wherein the ceftolozane active is provided as ceftolozane sulfate and the tazobactam is provided as tazobactam sodium.

11. The method of claim 2, wherein the pharmaceutical composition is administered intravenously over one hour.

12. The method of claim 1, wherein
(a) the 500 mg of ceftolozane active is co-administered with the 250 mg tazobactam in a first single pharmaceutical composition;
(b) the 100 mg of ceftolozane active is co-administered with the 50 mg tazobactam in a second single pharmaceutical composition;
(c) the ceftolozane active is provided as ceftolozane sulfate and the tazobactam active is provided as tazobactam sodium;
(d) the first pharmaceutical composition is obtained by reconstituting a mixture comprising ceftolozane sulfate and tazobactam sodium with sterile water for injection or 0.9% Sodium Chloride for injection, USP to form a reconstituted solution and withdrawing at least a portion of the reconstituted solution and adding it to an infusion bag containing 0.9% Sodium Chloride for Injection, USP or 5% Dextrose Injection, USP;
(e) the first pharmaceutical composition and the second pharmaceutical compositions are each separately administered intravenously over one hour;
(f) the complicated intra-abdominal infection is caused by a microorganism selected from the group consisting of: Citrobacter freundii, Enterobacter cloacae, Escherichia coli, Escherichia coli CTX-M-14 extended spectrum beta-lactamase producing strains, Escherichia coli CTX-M-15 extended spectrum beta-lactamase producing strains, Klebsiella oxytoca, Klebsiella pneumonia, Klebsiella pneumoniae CTX-M-15 extended spectrum beta-lactamase producing strains, Proteus mirabilis, Pseudomonas aeruginosa, Bacteroides fragilis, Bacteroides ovatus, Bacteroides thetaiotaomicron, Bacteroides vulgatus, Streptococcus anginosus, Streptococcus constellatus, and Streptococcus salivarius; or
(g) the complicated urinary tract infection is caused by one of the following Gram-negative microorganisms: Escherichia coli, Escherichia coli levofloxacin resistant strains, Escherichia coli CTX-M-14 extended spectrum beta-lactamase producing strains, Escherichia coli CTX-M-15 extended spectrum beta-lactamase producing strains, Klebsiella pneumoniae, Klebsiella pneumonia levofloxacin resistant strains, Klebsiella pneumonia CTX-M-15 extended spectrum beta-lactamase producing strains, Proteus mirabilis or Pseudomonas aeruginosa.

13. A method of treating a complicated intra-abdominal infection or a complicated urinary tract infection in a human patient with end stage renal disease with a ceftolozane/tazobactam composition that includes ceftolozane or a pharmaceutically acceptable salt thereof combined in a fixed dose ratio with tazobactam or a pharmaceutically acceptable salt thereof in an amount providing a 2:1 weight ratio between the amount of ceftolozane active and the amount of tazobactam active in the ceftolozane/tazobactam composition, where the method comprises intravenously administering to the human patient a single loading dose comprising 750 mg of the ceftolozane/tazobactam composition followed by a maintenance dose of the antibiotic composition comprising 150 mg of the ceftolozane/tazobactam composition.

14. The method of claim 13, wherein the infection is a complicated urinary tract infection and the method further comprises repeatedly administering the 150 mg maintenance dose of the ceftolozane/tazobactam composition every 8 hours for up to a total of 7 days.

15. The method of claim 13, wherein the infection is a complicated intra-abdominal infection and the method further comprises repeatedly administering the 150 mg maintenance dose of the ceftolozane/tazobactam composition every 8 hours for a total of 4-14 days.

16. The method of claim 15, wherein the method further comprises administering to the patient a therapeutically effective amount of metronidazole to the human patient separately from the ceftolozane and tazobactam.

17. The method of claim 13, wherein the ceftolozane active is provided as ceftolozane sulfate and the tazobactam is provided as tazobactam sodium.

18. The method of claim 13, wherein
(a) the ceftolozane active is provided as ceftolozane sulfate and the tazobactam active is provided as tazobactam sodium;
(b) the loading dose is obtained by reconstituting a mixture comprising ceftolozane sulfate and tazobactam sodium with sterile water for injection or 0.9% Sodium Chloride for injection, USP to form a reconstituted solution and withdrawing at least a portion of the reconstituted solution and adding it to an infusion bag containing 100 mL of 0.9% Sodium Chloride for Injection, USP or 5% Dextrose Injection, USP;
(c) the loading dose and each maintenance dose are each separately administered intravenously over one hour;
(d) the complicated intra-abdominal infection is caused by a microorganism selected from the group consisting of: Citrobacter freundii, Enterobacter cloacae, Escherichia coli, Escherichia coli CTX-M-14 extended spectrum beta-lactamase producing strains, Escherichia coli CTX-M-15 extended spectrum beta-lactamase producing strains, Klebsiella oxytoca, Klebsiella pneumonia, Klebsiella pneumoniae CTX-M-15 extended spectrum beta-lactamase producing strains, Proteus mirabilis, Pseudomonas aeruginosa, Bacteroides fragilis, Bacteroides ovatus, Bacteroides thetaiotaomicron, Bacteroides vulgatus, Streptococcus anginosus, Streptococcus constellatus, and Streptococcus salivarius; or
(e) the complicated urinary tract infection is caused by one of the following Gram-negative microorganisms: Escherichia coli, Escherichia coli levofloxacin resistant strains, Escherichia coli CTX-M-14 extended spectrum beta-lactamase producing strains, Escherichia coli CTX-M-15 extended spectrum beta-lactamase producing strains, Klebsiella pneumoniae, Klebsiella pneumonia levofloxacin resistant strains, Klebsiella pneumonia CTX-M-15 extended spectrum beta-lactamase producing strains, Proteus mirabilis or Pseudomonas aeruginosa.

19. A method of treating a complicated intra-abdominal infection or a complicated urinary tract infection in a human patient having a creatinine clearance of less than 15 mL/minute, the method comprising intravenously administering to the patient a first pharmaceutical composition comprising 500 mg of ceftolozane active and 250 mg tazobactam active, followed by administering one or more additional doses of a second pharmaceutical composition comprising 100 mg ceftolozane active and 50 mg of tazobactam active to the patient every 8 hours for the duration of a treatment period.

20. The method of claim 19, wherein the ceftolozane active is administered as reconstituted ceftolozane sulfate in a pharmaceutically acceptable carrier, and the tazobactam active is administered as reconstituted tazobactam sodium in a pharmaceutically acceptable carrier.

21. The method of claim 19, wherein the human patient has end stage renal disease and is on hemodialysis.

22. (canceled)

23. (canceled)

24. A method of treating a complicated intra-abdominal infection or a complicated urinary tract infection in a human patient having a creatinine clearance of less than 15 mL/minute, the method comprising intravenously administering to the patient a first liquid pharmaceutical composition comprising 500 mg of a compound of formula (I) 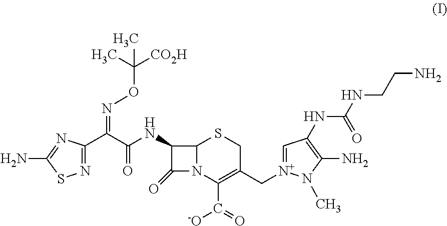
and 250 mg of a compound of formula (II) 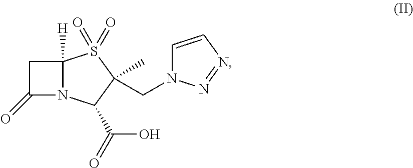 [SMILES: C[C@]1(Cn2ccnn2)[C@H](C(=O)O)N2C(=O)C[C@H]2S1(=O)=O]
followed by administering one or more additional doses of a second liquid pharmaceutical composition comprising 100 mg the compound of formula (I) and 50 mg of the compound of formula (II) to the patient every 8 hours for the duration of a treatment period.

25. (canceled)

26. The method of claim 24, wherein the compound of formula (I) is administered as a reconstituted sulfate salt of the compound of formula (I).

27. The method of claim 24, wherein the compound of formula (II) is administered as a reconstituted sodium salt of the compound of formula (II).

28. The method of claim 24, wherein the infection is an intra-abdominal infection.

29. The method of claim 28, further comprising administering metronidazole to the patient.

30. (canceled)

31. The method of claim 24, wherein the compound of formula (I) is administered as a sulfate salt of the compound of formula (I).

32. The method of claim 31, wherein the compound of formula (II) is administered as a sodium salt of the compound of formula (II).

33. The method of claim 32, wherein the method further comprises reconstituting a composition comprising the compound of formula (I) to obtain the first liquid pharmaceutical composition.

34. The method of claim 33, wherein the compound of formula (I) and the compound of formula (II) in the first liquid pharmaceutical composition are reconstituted from a unit dosage form containing 1,000 mg of ceftolozane active and 500 mg of tazobactam active prior to intravenous administration.
